(19) United States
(12) Patent Application Publication
(10) Pub. No.: US 2026/0240914 A1
(43) Pub. Date: Aug. 20, 2026

(54) MENIN-MLL INHIBITORS AND COMPOSITIONS FOR PROLIFERATION OF BETA CELLS

(71) Applicant: BIOMEA FUSION, INC., Redwood City, CA (US)

(72) Inventors: Mini BALAKRISHNAN, Redwood City, CA (US); Priyanka SOMANATH, Redwood City, CA (US); Thomas BUTLER, Redwood City, CA (US); Thorsten A. KIRSCHBERG, Redwood City, CA (US); James T. PALMER, Redwood City, CA (US)

(21) Appl. No.: 18/879,030

(22) PCT Filed: Jun. 28, 2023
(86) PCT No.: PCT/US2023/026503

§ 371 Date: Dec. 26, 2024

Related U.S. Application Data
(60) Provisional application No. 63/356,393, filed on Jun. 28, 2022.

(51) Int. Cl. A61K 35/39; A61K 31/506; A61K 31/519; A61K 31/5377; C12N 5/071

(52) U.S. Cl. CPC A61K 35/39; A61K 31/506; A61K 31/519; A61K 31/5377; C12N 5/0676; C12N 2501/999

(57) ABSTRACT

Methods and compositions using novel menin-MLL inhibitors enhancing proliferation of pancreatic beta cells to increase the efficacy of cell-based therapeutics are disclosed. Also provided are cell-based therapy methods and compositions.

### CROSS-REFERENCE

This application claims the benefit of U.S. Provisional Application No. 63/356,393, filed on Jun. 28, 2022, which is incorporated herein by reference in its entirety.

### FIELD OF THE INVENTION

Provided herein are methods relating to use of menin-MLL inhibitors in cell-based therapies. The methods and compositions of the disclosure are useful, for example, for the preparation of pancreatic beta cell compositions and their use for the treatment of pancreatic diseases and disorders, such as diabetes mellitus.

### BACKGROUND

Diabetes mellitus, commonly referred to as diabetes, is a major, worldwide medical problem. As of 2019, an estimated 463 million people had diabetes worldwide, with type 2 diabetes mellitus making up about 90% of the cases. IDF Diabetes Atlas Ninth Edition, 2019. Rates are similar in both women and men. Current and proposed treatments include insulin (type 1 diabetes), and metformin, sulfonylureas, acarbose, dipeptidyl peptidase-4 inhibitors, sitagliptin, thiazolidinedione, and SGLT2 inhibitors (type 2 diabetes). Even with these treatments, disease rates are rising, and the global economic cost of diabetes mellitus was estimated at $727 billion USD in 2017.

One potential opportunity for therapy is replacement of pancreatic beta cells in patients. The proposed therapy is based on the hypothesis that the replaced beta cells can restore glycemic control in patients. However, efforts to enhance proliferation of beta cells have shown limited success to date. Hayek & King, 2016, Clin. Diabetes and Endocrinol. 2:4. Therefore, more effective techniques for proliferation of beta cells are needed to enable cell replacement therapy.

### SUMMARY OF INVENTION

In several aspects, provided herein are methods and compositions for the enhancement of pancreatic beta cell proliferation and/or therapy with menin-MLL inhibitors. In certain embodiments, the menin-MLL inhibitors enhance proliferation of pancreatic beta cells in vivo prior to harvesting. In certain embodiments, the menin-MLL inhibitors enhance proliferation of pancreatic beta cells ex vivo. In certain embodiments, the menin-MLL inhibitors augment the benefits of receiving an infusion of pancreatic beta cells to a patient in need thereof. The methods are useful, for example, for the treatment of pancreatic diseases and disorders, such as diabetes mellitus. While not intending to be bound by a theory of operation, the methods are based, in part, on the discovery that menin-MLL inhibitors can enhance the proliferation of pancreatic beta cells.

In one aspect, provided herein are methods of enhancing proliferation of pancreatic beta cells in vivo with a menin-MLL inhibitor. In another aspect, provided herein are methods of enhancing proliferation of pancreatic beta cells ex vivo with a menin-MLL inhibitor. In another aspect, provided herein are pancreatic beta cells enhanced by methods provided herein. In another aspect, provided herein are compositions comprising the pancreatic beta cells. In another aspect, provided herein are methods of treatment comprising administration of the pancreatic beta cells or compositions. In another aspect, provided herein are methods of enhancing administration of pancreatic beta cells or compositions by administration of a menin-MLL inhibitor in combination. In yet another aspect, any or all of these aspects are combined.

Useful menin-MLL inhibitors include compounds that inhibit the activity of menin-MLL. In some embodiments, the inhibitor is a reversible inhibitor of menin-MLL interaction. In some embodiments, the inhibitor is an irreversible inhibitor of menin-MLL interaction. In some embodiments, the inhibitor is an irreversible inhibitor of menin-MLL interaction that form a covalent bond with a cysteine residue on menin. In some embodiments, the inhibitor forms a covalent bond with a Cys329 residue on menin. In certain embodiments, the inhibitor is an irreversible inhibitor of menin-MILL interaction is a compound according to Formula (I) having the structure:

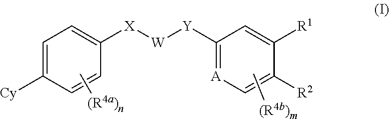

or a pharmaceutically acceptable salt thereof,
wherein:
A is C or N;
Cy is substituted or unsubstituted

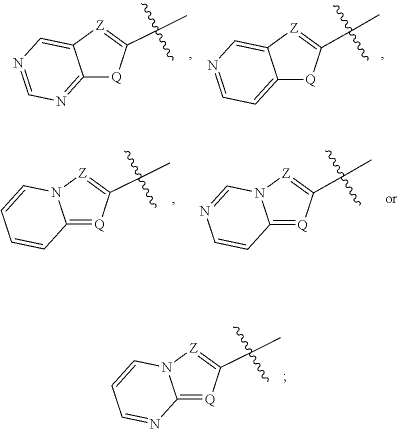

Q is N, —N(H)—, —O—, or —S—;
Z is —CR^{5a}═ or —N═;
X is —NR^{3a}—, —C(R^{3b})₂—, or —O—;
Y is a single bond, —NR^{3a}—, —C(R^{3b})₂—, or —O—;
W is —C(O)—, —S(O)—, or —S(O)₂—;
one of R¹ and R² is Cy²-N(H)C(O)—C(R^{6a})═C(R^{6b})(R^{6c}), or CH₂—Cy²-N(H)C(O)—C(R^{6a})═C(R^{6b})(R^{6c}); and other is H, C₁₋₆ alkyl, C₁₋₆ haloalkyl, halo, or CN;
Cy² is an optionally substituted group selected from phenyl, pyridyl, or a 4-7 membered heterocycloalkyl ring having 1-2 heteroatoms independently selected from nitrogen, oxygen, or sulfur;
each R^{3a}, and R^{3b} is independently H or C₁₋₆ alkyl;
each R^{4a} and R^{4b} is independently H, halo, CN, OR, —N(R)₂, —C(O)N(R)₂, —NRC(O)R, —SO₂R, —C(O)R, —CO₂R, or an optionally substituted group selected from C₁₋₆ alkyl, C₃₋₇ cycloalkyl, a 4-7 membered heterocycloalkyl ring having 1-2 heteroatoms independently selected from nitrogen, oxygen, or sulfur, phenyl, an 8-10 membered bicyclic aryl ring, and a 5-6 membered heteroaryl ring having 1-4 heteroatoms independently selected from nitrogen, oxygen, or sulfur;
each R is independently H, or an optionally substituted group selected from C₁₋₆ aliphatic, phenyl, an 8-10 membered bicyclic aryl ring, a 4-7 membered saturated or partially unsaturated heterocyclic ring having 1-2 heteroatoms independently selected from nitrogen, oxygen, or sulfur, and a 5-6 membered heteroaryl ring having 1-4 heteroatoms independently selected from nitrogen, oxygen, or sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, or sulfur;
R^{5a} is H, C₁₋₆ alkyl, C₁₋₆ haloalkyl, halo, or CN;
each R^{6a} and R^{6b} is independently H or C₁₋₆ alkyl; or R^{6a} and R^{6b} are joined together to form a bond;
R^{6c} is H or substituted or unsubstituted C₁₋₆ alkyl;
m is 1, 2, or 3; and n is 1, 2, 3, or 4.
   In certain embodiments, the menin-MLL inhibitor is selected from compounds 1-26 described herein.

In one aspect, provided herein are proliferation methods, compositions, and cell therapy methods for treating a condition in a patient in need thereof. In certain embodiments, the methods comprise administering a menin-MLL inhibitor to a donor under conditions suitable to promote activation or proliferation of pancreatic beta cells in the donor. In certain embodiments, the methods further comprise harvesting circulating pancreatic beta cells from the donor. In certain embodiments, the methods further comprise expanding pancreatic beta cells from said donor ex vivo. In some embodiments, the proliferation step is in the presence of one or more menin-MLL inhibitors. In certain embodiments, the methods further comprise administering the pancreatic beta cells to a patient in need thereof. In some embodiments, the administering is in combination with one or more menin-MLL inhibitors.

In another aspect, provided herein are proliferation methods, compositions, and cell therapy methods for treating a condition in a patient in need thereof. In certain embodiments, the methods comprise harvesting beta cells from a donor. In certain embodiments, the methods comprise administering a menin-MLL inhibitor to a donor under conditions suitable to promote activation or proliferation of pancreatic beta cells in the donor. In certain embodiments, the methods further comprise expanding pancreatic beta cells ex vivo. In certain embodiments, the methods further comprise administering the pancreatic beta cells to a patient in need thereof.

In another aspect, provided herein are proliferation methods, compositions, and cell therapy methods for treating a condition in a patient in need thereof. In certain embodiments, the methods comprise harvesting pancreatic beta cells from a donor. In certain embodiments, the methods further comprise expanding pancreatic beta cells ex vivo. In certain embodiments, the proliferation step is in the presence of a menin-MLL inhibitor. In certain embodiments, the methods further comprise administering the pancreatic beta cells to a patient in need thereof.

In another aspect, provided herein are proliferation methods, compositions, and cell therapy methods for treating a condition in a patient in need thereof. In certain embodiments, the methods comprise harvesting pancreatic beta cells from a donor. In certain embodiments, the methods further comprise expanding pancreatic beta cells ex vivo. In certain embodiments, the methods further comprise administering the pancreatic beta cells to a patient in need thereof in combination with administering a sufficient amount of a menin-MLL inhibitor, for instance to enhance effectiveness of the pancreatic beta cells.

In certain aspects, any of the above methods are combined. In certain embodiments, the methods comprise administering a menin-MLL inhibitor to a donor prior to harvesting, and administering expanded pancreatic beta cells to a patient in need thereof in combination with a menin-MLL inhibitor. In certain embodiments, the methods comprise proliferation in the presence of a menin-MLL inhibitor, and administering expanded pancreatic beta cells to a patient in need thereof in combination with a menin-MLL inhibitor.

The pancreatic beta cells can be any pancreatic beta cells deemed useful to the person of skill. In certain embodiments, embodiments, the pancreatic beta cells can be isolated from the pancreas of a donor. In certain embodiments, the donor is from the same species as the subject. In certain embodiments, the donor is of a species different from the subject. In certain embodiments, the donor is porcine, and the subject is human. In certain embodiments, the donor is human, and the subject is human. In certain embodiments, the donor is the patient. In certain embodiments, the donor is not the patient. In certain embodiments, the cells are human pancreatic beta cells. In certain embodiments, the cells are human neonatal pancreatic beta cells. In certain embodiments, the cells are stem cell derived pancreatic beta cells. In certain embodiments, the pancreatic beta cells are selected from cultured beta cell preparations, encapsulated beta cell preparations, porcine beta cells, transgenic porcine beta cells, human beta cells, genetically modified human beta cells, non-human primate beta cells, genetically modified non-human primate beta cells, porcine beta islet cells, genetically modified porcine beta islet cells, fetal human beta cells, genetically modified fetal human beta cells, pig islet clusters, and genetically modified pig islet clusters.

In another aspect, provided herein are methods of expanding pancreatic beta cells in vitro. In certain embodiments, the methods comprise expanding pancreatic beta cells in vitro in the presence of a menin-MILL inhibitor.

In another aspect, provided herein are compositions comprising cells made by any of the methods described herein. The compositions of the methods described herein can be used, for example, for the treatment of pancreatic diseases and disorders, such as diabetes mellitus. In certain embodiments, cells made by the methods described herein can be used in the treatment of diabetes mellitus.

### BRIEF DESCRIPTION OF THE FIGURES

FIG. 1 provides induction of proliferation of human pancreatic beta cells by compound 10 described herein at 14 days of culturing in presence of compound 10 (0.3 μM). A is ATP content; B is proliferating beta cell fraction; C is beta cell fraction. Vehicle control (DMSO) also shown.

FIG. 2 provides induction of proliferation of human pancreatic beta cells by compound 10 described herein at 21 days of culturing in presence of compound 10 (0.3 μM). A is ATP content; B is proliferating beta cell fraction; C is beta cell fraction. Vehicle control (DMSO) also shown.

FIG. 3 provides induction of proliferation of human pancreatic beta cells by compound 10 described herein at 7 days of culturing in presence of compound 10 (0.075 μM, 0.0150 M, and 0.3 μM). DMSO control also shown.

FIG. 4 provides induction of proliferation of human pancreatic beta cells by compound 10 described herein at 14 days of culturing in presence of compound 10 (0.075 μM, 0.0150 M, and 0.3 M). DMSO control also shown.

FIG. 5 provides induction of proliferation of human pancreatic beta cells by compound 10 described herein at 21 days of culturing in presence of compound 10 (0.075 μM, 0.0150 M, and 0.3 M). DMSO control also shown.

FIG. 6 shows ATP content as a measure of cell viability of human islet microtissues cultured in presence of compound 10 (0.075 μM, 0.15 μM and 0.3 μM) in standard media (5.5 mM glucose) or high glucose media (8 mM glucose) for 1, 2 or 3 weeks. DMSO control is also shown.

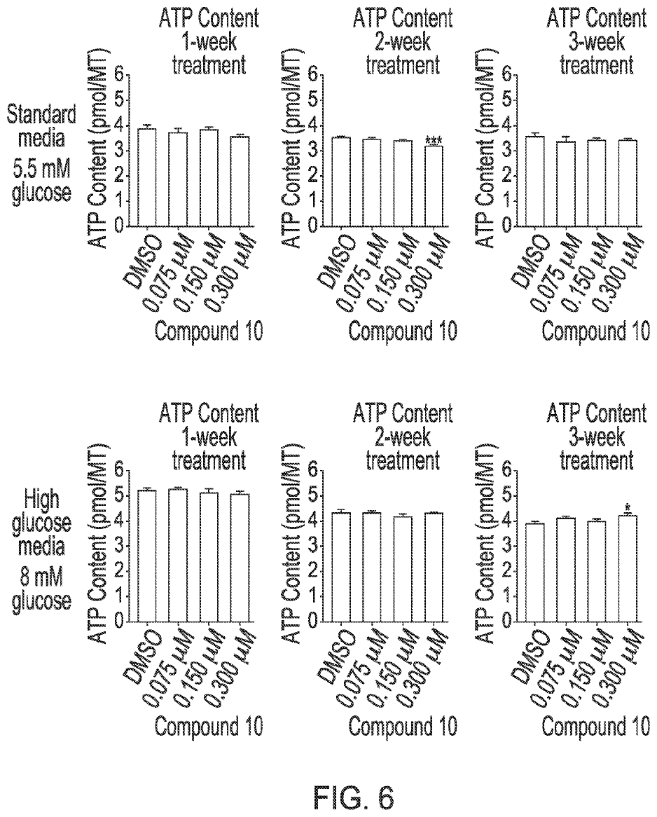

FIG. 7 shows insulin content in human islet beta cells cultured in presence of compound 10 (0.075 μM, 0.15 μM and 0.3 μM) in standard media (5.5 mM glucose) or high glucose media (8 mM glucose) for 1, 2 or 3 weeks. DMSO control is also shown.

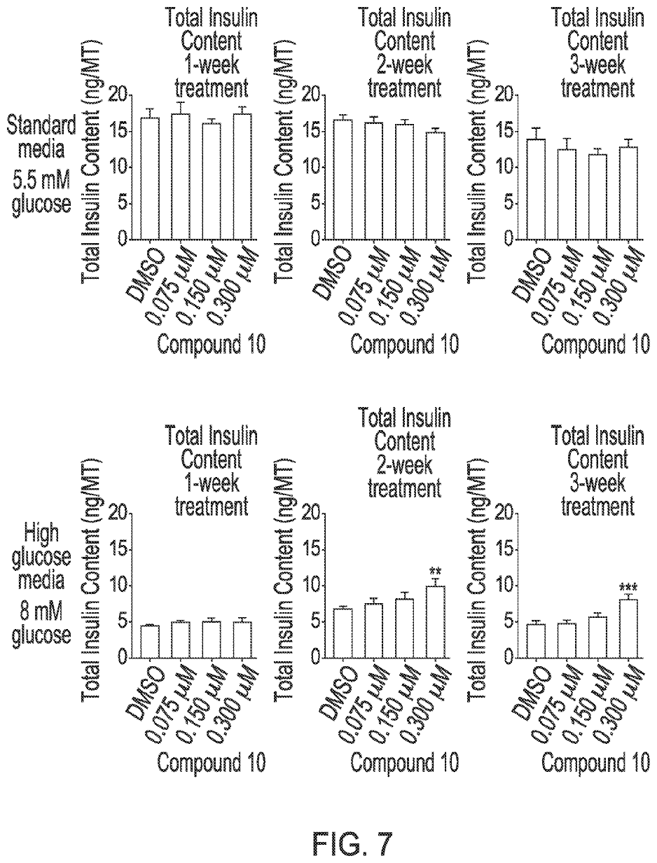

FIG. 8 shows glucose-stimulated insulin secretion from human islet beta cells cultured in presence of compound 10 (0.075 μM, 0.15 μM and 0.3 μM) in standard media (5.5 mM glucose) or high glucose media (8 mM glucose) for 1 or 2 weeks. DMSO control is also shown.

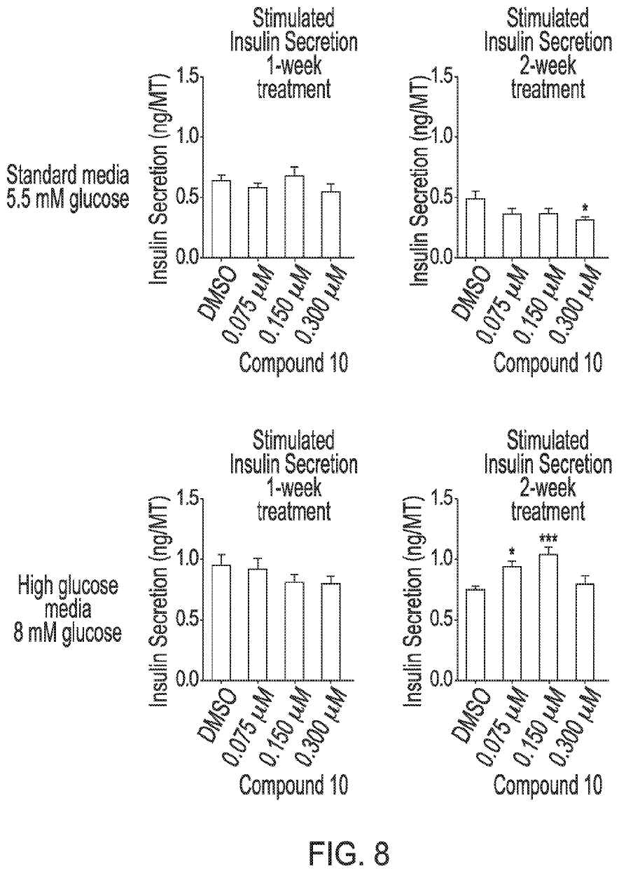

### DETAILED DESCRIPTION

### Definitions

Unless defined otherwise, all technical and scientific terms used herein have the same meaning as is commonly understood by one of skill in the art to which the claimed subject matter belongs. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. Use of the term “including” as well as other forms, such as “include”, “includes,” and “included,” is not limiting. Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

All publications and patents mentioned herein are incorporated herein by reference in their entirety for the purpose of describing and disclosing, for example, the constructs and methodologies that are described in the publications, which might be used in connection with the methods, compositions and compounds described herein. The publications discussed herein are provided solely for their disclosure prior to the filing date of the present application. Nothing herein is to be construed as an admission that the inventors described herein are not entitled to antedate such disclosure by virtue of prior invention or for any other reason.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In some embodiments, an alkyl comprises five to fifteen carbon atoms (e.g., C₅-C₁₅ alkyl). In certain embodiments, an alkyl comprises five to eight carbon atoms (e.g., C₅-C₈ alkyl). The alkyl is attached to the rest of the molecule by a single bond, for example, methyl (Me), ethyl (Et), n-propyl (n-pr), 1-methylethyl (iso-propyl or i-Pr), n-butyl (n-Bu), n-pentyl, 1,1-dimethylethyl (t-butyl, or t-Bu), 3-methylhexyl, 2-methylhexyl, and the like. Unless stated otherwise specifically in the specification, an alkyl group is optionally substituted as defined and described below and herein.

The alkyl group could also be a “lower alkyl” having 1 to 6 carbon atoms.

As used herein, C₁-Cₓ includes C₁-C₂, C₁-C₃, . . . C₁-Cₓ.

“Alkenyl” refers to a straight or branched hydrocarbon chain radical group consisting solely of carbon and hydrogen atoms, containing at least one double bond, and having from two to twelve carbon atoms. In certain embodiments, an alkenyl comprises two to eight carbon atoms. In some embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted as defined and described below and herein.

“Alkynyl” refers to a straight or branched hydrocarbon chain radical group consisting solely of carbon and hydrogen atoms, containing at least one triple bond, having from two to twelve carbon atoms. In certain embodiments, an alkynyl comprises two to eight carbon atoms. In some embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted as defined and described below and herein.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon in the alkylene chain or through any two carbons within the chain. Unless stated otherwise specifically in the specification, an alkylene chain is optionally substituted as defined and described below and herein.

“Alkenylene” or “alkenylene chain” refers to a straight or branched divalent hydrocarbon chain linking the rest of the molecule to a radical group, consisting solely of carbon and hydrogen, containing at least one double bond and having from two to twelve carbon atoms, for example, ethenylene, propenylene, n-butenylene, and the like. The alkenylene chain is attached to the rest of the molecule through a double bond or a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain is optionally substituted as defined and described below and herein.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Hückel theory. Aryl groups include, but are not limited to, groups such as phenyl (Ph), fluorenyl, and naphthyl. Unless stated otherwise specifically in the specification, the term “aryl” or the prefix “ar-” (such as in “aralkyl”) is meant to include aryl radicals optionally substituted as defined and described below and herein.

“Aralkyl” refers to a radical of the formula —R^{c}-aryl where R^{c} is an alkylene chain as defined above, for example, benzyl, diphenylmethyl and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In some embodiments, a carbocyclyl comprises five to seven carbon atoms. The carbocyclyl is attached to the rest of the molecule by a single bond. Carbocyclyl is optionally saturated, (i.e., containing single C—C bonds only) or unsaturated (i.e., containing one or more double bonds or triple bonds.) A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted as defined and described below and herein. “Halo” or “halogen” refers to bromo, chloro, fluoro or iodo substituents.

The terms “haloalkyl,” “haloalkenyl,” “haloalkynyl” and “haloalkoxy” include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In some embodiments in which two or more hydrogen atoms are replaced with halogen atoms, the halogen atoms are not all the same as one another.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical is optionally substituted as defined above for an alkyl group.

As used herein, the term “non-aromatic heterocycle”, “heterocycloalkyl” or “heteroalicyclic” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen and sulfur. The radicals may be fused with an aryl or heteroaryl. Heterocycloalkyl rings can be formed by three to 14 ring atoms, such as three, four, five, six, seven, eight, nine, or more than nine atoms. Heterocycloalkyl rings can be optionally substituted. In certain embodiments, non-aromatic heterocycles contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include:

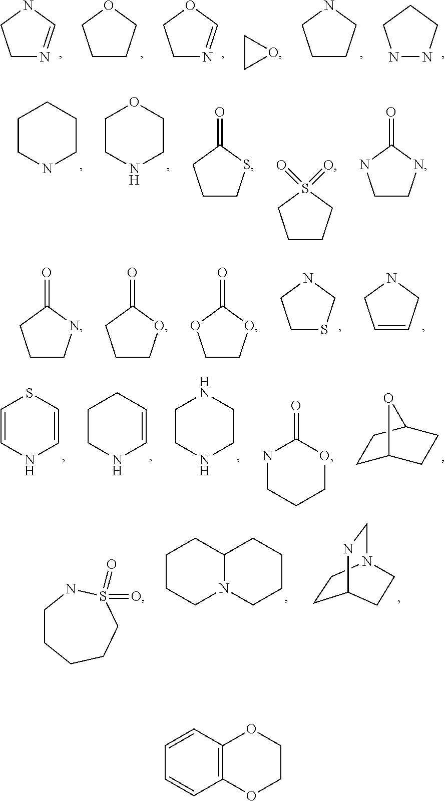

and the like. The term heteroalicyclic also includes all ring forms of the carbohydrates, including but not limited to the monosaccharides, the disaccharides and the oligosaccharides. Depending on the structure, a heterocycloalkyl group can be a monoradical or a diradical (i.e., a heterocycloalkylene group).

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. In some embodiments, heteroaryl rings have five, six, seven, eight, nine, or more than nine ring atoms. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted as defined and described below and herein.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Sulfanyl” refers to the —S— radical.

“Sulfinyl” refers to the —S(═O)— radical.

“Sulfonyl” refers to the —S(═O)₂— radical.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Imino” refers to the ═NH radical.

“Thioxo” refers to the ═S radical.

An “alkoxy” group refers to a (alkyl)O— group, where alkyl is as defined herein.

An “aryloxy” group refers to an (aryl)O— group, where aryl is as defined herein.

“Carbocyclylalkyl” means an alkyl radical, as defined herein, substituted with a carbocyclyl group. “Cycloalkylalkyl” means an alkyl radical, as defined herein, substituted with a cycloalkyl group. Non-limiting cycloalkylalkyl groups include cyclopropylmethyl, cyclobutylmethyl, cyclopentylmethyl, cyclohexylmethyl, and the like.

As used herein, the terms “heteroalkyl” “heteroalkenyl” and “heteroalkynyl” include optionally substituted alkyl, alkenyl and alkynyl radicals in which one or more skeletal chain atoms is a heteroatom, e.g., oxygen, nitrogen, sulfur, silicon, phosphorus or combinations thereof. The heteroatom(s) may be placed at any interior position of the heteroalkyl group or at the position at which the heteroalkyl group is attached to the remainder of the molecule. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃.

The term “heteroatom” refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

The term “bond,” “direct bond” or “single bond” refers to a chemical bond between two atoms, or two moieties when the atoms joined by the bond are considered to be part of larger substructure.

An “isocyanato” group refers to a —NCO group.

An “isothiocyanato” group refers to a —NCS group.

The term “moiety” refers to a specific segment or functional group of a molecule. Chemical moieties are often recognized chemical entities embedded in or appended to a molecule.

A “thioalkoxy” or “alkylthio” group refers to a —S-alkyl group.

A “alkylthioalkyl” group refers to an alkyl group substituted with a —S-alkyl group.

As used herein, the term “acyloxy” refers to a group of formula RC(═O)O—.

“Carboxy” means a —C(O)OH radical.

As used herein, the term “acetyl” refers to a group of formula —C(═O)CH₃.

“Acyl” refers to the group —C(O)R.

As used herein, the term “trihalomethanesulfonyl” refers to a group of formula X₃CS(═O)₂— where X is a halogen.

“Cyanoalkyl” means an alkyl radical, as defined herein, substituted with at least one cyano group.

As used herein, the term “N-sulfonamido” or “sulfonylamino” refers to a group of formula RS(═O)₂NH—.

As used herein, the term “O-carbamyl” refers to a group of formula —OC(═O)NR₂.

As used herein, the term “N-carbamyl” refers to a group of formula ROC(═O)NH—.

As used herein, the term “O-thiocarbamyl” refers to a group of formula —OC(═S)NR₂.

As used herein, “N-thiocarbamyl” refers to a group of formula ROC(═S)NH—.

As used herein, the term “C-amido” refers to a group of formula —C(═O)NR₂.

“Aminocarbonyl” refers to a —CONH₂ radical.

As used herein, the term “N-amido” refers to a group of formula RC(═O)NH—.

“Hydroxyalkyl” refers to an alkyl radical, as defined herein, substituted with at least one hydroxy group. Non-limiting examples of a hydroxyalkyl include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl.

“Alkoxyalkyl” refers to an alkyl radical, as defined herein, substituted with an alkoxy group, as defined herein.

An “alkenyloxy” group refers to a (alkenyl)O— group, where alkenyl is as defined herein.

The term “alkylamine” refers to the —N(alkyl)ₓH_{y} group, where x and y are selected from among x=1, y=1 and x=2, y=0. When x=2, the alkyl groups, taken together with the N atom to which they are attached, can optionally form a cyclic ring system.

“Alkylaminoalkyl” refers to an alkyl radical, as defined herein, substituted with an alkylamine, as defined herein.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). An amide moiety may form a linkage between an amino acid or a peptide molecule and a compound described herein, thereby forming a prodrug. Any amine, or carboxyl side chain on the compounds described herein can be amidified. The procedures and specific groups to make such amides are known to those of skill in the art and can readily be found in reference sources such as Greene and Wuts, Protective Groups in Organic Synthesis, 3^{rd} Ed., John Wiley & Sons, New York, NY, 1999, which is incorporated herein by reference in its entirety.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3^{rd} Ed., John Wiley & Sons, New York, NY, 1999, which is incorporated herein by reference in its entirety.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

As used herein, the term “ring system” refers to one, or more than one ring.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “fused” refers to structures in which two or more rings share one or more bonds.

As described herein, compounds of the invention may be “optionally substituted”. In general, the term “substituted,” whether preceded by the term “optionally” or not, means that one or more hydrogens of a designated moiety is/are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)_{0- 4}N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}, —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C_{1- 4} straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “nucleophile” or “nucleophilic” refers to an electron rich compound, or moiety thereof.

The term “electrophile” or “electrophilic” refers to an electron poor or electron deficient molecule, or moiety thereof. Examples of electrophiles include, but in no way are limited to, Michael acceptor moieties.

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

As used herein, “amelioration” of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

“Bioavailability” refers to the percentage of the weight of compounds disclosed herein, such as, compounds of any of Formula (I)—(XLIIIc) dosed that is delivered into the general circulation of the animal or human being studied. The total exposure (AUC_{(0-∞)}) of a drug when administered intravenously is usually defined as 100% bioavailable (F %). “Oral bioavailability” refers to the extent to which compounds disclosed herein, such as, compounds of any of Formula (I)—(XLIIIc) are absorbed into the general circulation when the pharmaceutical composition is taken orally as compared to intravenous injection.

“Blood plasma concentration” refers to the concentration of compounds disclosed herein, such as, compounds of any of Formula (I)—(XLIIIc) in the plasma component of blood of a subject. It is understood that the plasma concentration of compounds of any of Formula (I)—(XLIIIc) may vary significantly between subjects, due to variability with respect to metabolism and/or possible interactions with other therapeutic agents. In accordance with some embodiments disclosed herein, the blood plasma concentration of the compounds of any of Formula (I)—(XLIIIc) may vary from subject to subject. Likewise, values such as maximum plasma concentration (Cₘₐₓ) or time to reach maximum plasma concentration (Tₘₐₓ), or total area under the plasma concentration time curve (AUC_{(0-∞)}) may vary from subject to subject. Due to this variability, the amount necessary to constitute “a therapeutically effective amount” of a compound of any of Formula (I)—(XLIIIc) may vary from subject to subject.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of the compound of any of Formula (I)—(XVII), age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

The terms “enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder or condition. An “enhancing-effective amount,” as used herein, refers to an amount adequate to enhance the effect of a therapeutic agent in the treatment of a disease, disorder or condition. When used in a patient, amounts effective for this use will depend on the severity and course of the disease, disorder or condition, previous therapy, the patient's health status and response to the drugs, and the judgment of the treating physician.

The term “identical,” as used herein, refers to two or more sequences or subsequences which are the same. In addition, the term “substantially identical,” as used herein, refers to two or more sequences which have a percentage of sequential units which are the same when compared and aligned for maximum correspondence over a comparison window, or designated region as measured using comparison algorithms or by manual alignment and visual inspection. By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages to describe the “percent identity” of two or more sequences. The identity of a sequence can exist over a region that is at least about 75-100 sequential units in length, over a region that is about 50 sequential units in length, or, where not specified, across the entire sequence. This definition also refers to the complement of a test sequence. By way of example only, two or more polypeptide sequences are identical when the amino acid residues are the same, while two or more polypeptide sequences are “substantially identical” if the amino acid residues are about 60% identical, about 65% identical, about 70% identical, about 75% identical, about 80% identical, about 85% identical, about 90% identical, or about 95% identical over a specified region. The identity can exist over a region that is at least about 75-100 amino acids in length, over a region that is about 50 amino acids in length, or, where not specified, across the entire sequence of a polypeptide sequence. In addition, by way of example only, two or more polynucleotide sequences are identical when the nucleic acid residues are the same, while two or more polynucleotide sequences are “substantially identical” if the nucleic acid residues are about 60% identical, about 65% identical, about 70% identical, about 75% identical, about 80% identical, about 85% identical, about 90% identical, or about 95% identical over a specified region. The identity can exist over a region that is at least about 75-100 nucleic acids in length, over a region that is about 50 nucleic acids in length, or, where not specified, across the entire sequence of a polynucleotide sequence.

The term “isolated,” as used herein, refers to separating and removing a component of interest from components not of interest. Isolated substances can be in either a dry or semi-dry state, or in solution, including but not limited to an aqueous solution. The isolated component can be in a homogeneous state or the isolated component can be a part of a pharmaceutical composition that comprises additional pharmaceutically acceptable carriers and/or excipients. By way of example only, nucleic acids or proteins are “isolated” when such nucleic acids or proteins are free of at least some of the cellular components with which it is associated in the natural state, or that the nucleic acid or protein has been concentrated to a level greater than the concentration of its in vivo or in vitro production. Also, by way of example, a gene is isolated when separated from open reading frames which flank the gene and encode a protein other than the gene of interest.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes, such as, oxidation reactions) by which a particular substance is changed by an organism. Thus, enzymes may produce specific structural alterations to a compound. For example, cytochrome P450 catalyzes a variety of oxidative and reductive reactions while uridine diphosphate glucuronyl transferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art. In some embodiments, metabolites of a compound are formed by oxidative processes and correspond to the corresponding hydroxy-containing compound. In some embodiments, a compound is metabolized to pharmacologically active metabolites.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

As used herein, the term “modulator” refers to a compound that alters an activity of a molecule. For example, a modulator can cause an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

The term “irreversible inhibitor,” as used herein, refers to a compound that, upon contact with a target protein (e.g., menin) causes the formation of a new covalent bond with or within the protein, whereby one or more of the target protein's biological activities (e.g., phosphotransferase activity) is diminished or abolished notwithstanding the subsequent presence or absence of the irreversible inhibitor. In contrast, a reversible inhibitor compound upon contact with a target protein does not cause the formation of a new covalent bond with or within the protein and therefore can associate and dissociate from the target protein.

The term “irreversible inhibitor of menin-MLL protein-protein interaction” as used herein, refers to an inhibitor of menin that can form a covalent bond with an amino acid residue of menin. In one or more embodiments, the irreversible inhibitor of menin can form a covalent bond with a Cys residue of menin; in particular embodiments, the irreversible inhibitor can form a covalent bond with a Cys 329 residue (or a homolog thereof) of menin.

The term “prophylactically effective amount,” as used herein, refers that amount of a composition applied to a patient that will relieve to some extent one or more of the symptoms of a disease, condition, or disorder being treated. In such prophylactic applications, such amounts may depend on the patient's state of health, weight, and the like. It is considered well within the skill of the art for one to determine such prophylactically effective amounts by routine experimentation, including, but not limited to, a dose escalation clinical trial.

As used herein, the term “selective binding compound” refers to a compound that selectively binds to any portion of one or more target proteins.

As used herein, the term “selectively binds” refers to the ability of a selective binding compound to bind to a target protein, such as, for example, menin, with greater affinity than it binds to a non-target protein. In certain embodiments, specific binding refers to binding to a target with an affinity that is at least 10, 50, 100, 250, 500, 1000 or more times greater than the affinity for a non-target.

As used herein, the term “selective modulator” refers to a compound that selectively modulates a target activity relative to a non-target activity. In certain embodiments, specific modulator refers to modulating a target activity at least 10, 50, 100, 250, 500, 1000 times more than a non-target activity.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater.

The term “subject” or “patient” as used herein, refers to an animal which is the object of treatment, observation or experiment. By way of example only, a subject may be, but is not limited to, a mammal including, but not limited to, a human.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes, and amelioration of one or more symptoms associated with a disease or condition (e.g., diabetes mellitus).

As used herein, the term “target protein” refers to a molecule or a portion of a protein capable of being bound by a selective binding compound. In certain embodiments, a target protein is menin.

The terms “treat,” “treating,” or “treatment” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating,” or “treatment,” include, but are not limited to, prophylactic and/or therapeutic treatments.

As used herein, the IC₅₀ refers to an amount, concentration or dosage of a particular test compound that achieves a 50% inhibition of a maximal response, such as inhibition of menin-MLL, in an assay that measures such response.

As used herein, EC₅₀ refers to a dosage, concentration or amount of a particular test compound that elicits a dose-dependent response at 50% of maximal expression of a particular response that is induced, provoked, or potentiated by the particular test compound.

### Overview

Provided herein are methods and compositions using menin-MLL inhibitors for cell proliferation and/or therapy. In certain embodiments, the menin-MLL inhibitors enhance pancreatic beta cells in vivo prior to harvesting. In certain embodiments, the menin-MLL inhibitors enhance proliferation of pancreatic beta cells ex vivo. In certain embodiments, the menin-MLL inhibitors are administered to a patient to enhance administration of pancreatic beta cells to the same patient. The cells can be any pancreatic beta cells deemed useful by the practitioner of skill. In certain embodiments, the cells are human pancreatic beta cells. In certain embodiments, the cells are human neonatal pancreatic beta cells. In certain embodiments, the cells are human stem cell derived pancreatic beta cells.

Methods for cell proliferation and therapy are well known, and the steps provided herein can utilize standard techniques known to the practitioner of skill, unless specified otherwise. Generally, the methods comprise any or all of the following steps. One or more menin-MLL inhibitors can enhance one or more of the steps, as described in detail below.

In certain embodiments, pancreatic beta cells are harvested from the donor. In a second step, the harvested pancreatic beta cells are expanded. Following proliferation, expanded pancreatic beta cells are recovered. The pancreatic beta cells can be stored. In a further step, the recovered pancreatic beta cells are administered to a patient by standard techniques, for instance infusion. As described in detail below, one or more menin-MLL inhibitors can enhance growth, proliferation, and development of pancreatic beta cells in the host prior to harvesting, can enhance proliferation, can enhance administration of the pancreatic beta cells for therapy, and/or can enhance the pancreatic beta cells post-administration. Embodiments are described briefly in the following paragraphs and in more detail in the sections below.

The menin-MLL inhibitors are useful at one or more steps of the methods. In certain embodiments, a menin-MLL inhibitor is used in one step. In certain embodiments, menin-MLL inhibitors are used in more than one step. In certain embodiments, menin-MLL inhibitors are used in two steps. In certain embodiments, menin-MLL inhibitors are used in three steps. In certain embodiments, a menin-MLL inhibitor enhances proliferation of pancreatic beta cells in vivo in a donor. In certain embodiments, a menin-MLL inhibitor enhances proliferation of pancreatic beta cells ex vivo. In certain embodiments, a menin-MLL inhibitor enhances the post-administration effectiveness of the administered pancreatic beta cells in the patient. In certain embodiments, a menin-MLL inhibitor administered to a patient enhances the benefits of the administered pancreatic beta cells in the patient in need of such pancreatic beta cell therapy.

In certain embodiments, the methods comprise administering a menin-MLL inhibitor to an individual. The individual can be a donor of pancreatic beta cells. The donor can be an autologous donor and also a patient in need of therapy. The donor also can be an individual providing cells for allogeneic therapy. In certain embodiments, the menin-MLL inhibitor is administered to a donor prior to harvest of pancreatic beta cells. In certain embodiments, the menin-MLL inhibitor increases the overall number of pancreatic beta cells that can be harvested from the donor.

In certain embodiments, a menin-MLL inhibitor enhances proliferation of pancreatic beta cells. The pancreatic beta cells can be harvested according to standard techniques. In certain embodiments, the harvested pancreatic beta cells are cultured ex vivo in the presence of one or more menin-MLL inhibitors. In some embodiments, culture in the presence of the menin-MILL inhibitors results in increased yield of pancreatic beta cells that are harvested.

In certain embodiments, a menin-MLL inhibitor enhances administration of pancreatic beta cells to a recipient or patient in need thereof. In certain embodiments, a menin-MLL inhibitor is administered in combination with expanded pancreatic beta cells to an individual in need thereof. In certain embodiments, provided here are methods of cellular therapy in an individual in need thereof, comprising: administering a menin-MLL inhibitor to the individual in an amount effective to enhance the cellular therapy; and administering an effective amount of expanded pancreatic beta cells to the individual for cellular therapy. The menin-MLL inhibitor can be administered any time deemed suitable by the practitioner of skill. In certain embodiments, the menin-MLL inhibitor is administered prior to harvest, after harvest, prior to infusion, after infusion, or any combination thereof. In certain embodiments, the method is carried out ex vivo. In certain embodiments, the method is carried out in vivo. In one or more embodiments, the cells are human neonatal pancreatic beta cells. In one or more embodiments, the cells are stem cell derived beta cells.

In certain embodiments, provided herein is a method of increasing cell proliferation in a population of pancreatic beta cells. The method comprises contacting the population of pancreatic beta cells with a compound described herein (i.e., a compound of formula (I)) under conditions effective to increase cell proliferation in the population of pancreatic beta cells. In certain embodiments, contacting is carried out with a composition (i.e., a single composition) comprising the compound. In one or more embodiments, the cells are human neonatal pancreatic beta cells. In one or more embodiments, the cells are stem cell derived beta cells.

In certain embodiments, the methods may further comprise contacting the population of pancreatic beta cells with a transforming growth factor beta (TGF beta) superfamily signaling pathway inhibitor. In accordance with this embodiment, the method may be carried out with a composition comprising the compound and the TGF beta superfamily signaling pathway inhibitor. In another embodiment, the compound of Formula (I) and the TGF beta superfamily signaling pathway inhibitor separately contact a population of pancreatic beta cells simultaneously or in sequence. In certain embodiments, the method is carried out ex vivo. In certain embodiments, the method is carried out in vivo. In one or more embodiments, the cells are human neonatal pancreatic beta cells. In one or more embodiments, the cells are stem cell derived beta cells.

In certain embodiments, provided herein are methods of treating a subject for a condition associated with insufficient insulin secretion. The methods comprise administering to a subject in need of treatment for a condition associated with an insufficient level of insulin secretion the pancreatic beta cells proliferated produced according to a method described herein. In one or more embodiments, the cells are human neonatal pancreatic beta cells. In one or more embodiments, the cells are stem cell derived beta cells.

In certain embodiments, provided herein are methods of treating a subject for a condition associated with insufficient insulin secretion, the methods comprising: i) contacting a population of pancreatic beta cells, with a compound according to formula I to increase cell proliferation in population of pancreatic beta cells; and ii) administering the proliferated beta cells to the subject. In one or more embodiments, the cells are human pancreatic beta cells. In one or more embodiments, the cells are human neonatal pancreatic beta cells. In one or more embodiments, the cells are stem cell derived beta cells.

Useful techniques are described in Valdes-Gonzalez et al., 2010, Clin. Exp. Immunol. 162:537-542; Elliott et al., 2000, Cell Transplant. 9:895-901; Elliott et al, 2007, Xenotransplantation 14:157-161; and Hering et al., 2006, Nat Med 12:301-303; Memon and Abdelalim, 2020, Cells 9(2):283; the contents of each of which are hereby incorporated by reference in their entireties.

### Menin-MLL Inhibitors

In certain embodiments, the menin-MLL inhibitor is a compound according to Formula (I) having the structure:

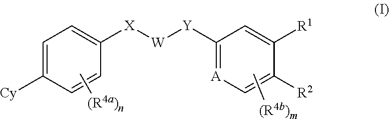

or a pharmaceutically acceptable salt thereof,
wherein:
A is C or N;
Cy is substituted or unsubstituted

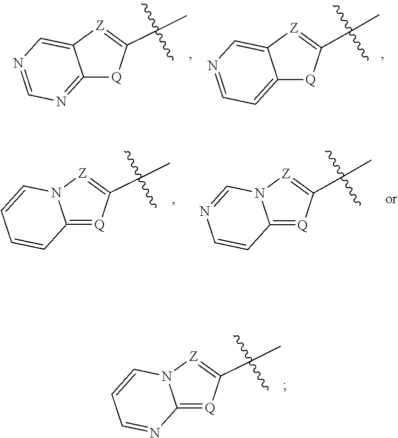

Q is N, —N(H)—, —O—, or —S—;
Z is —CR₅ₐ═ or —N═;
X is —NR^{3a}—, —C(R^{3b})₂—, or —O—;
Y is a single bond, —NR^{3a}—, —C(R^{3b})₂—, or —O—;
W is —C(O)—, —S(O)—, or —S(O)₂—;
one of R¹ and R² is Cy²-N(H)C(O)—C(R^{6a})═C(R^{6b})(R^{6c}), or CH₂—Cy²-N(H)C(O)—C(R^{6a})═C(R^{6b})(R^{6c}); and other is H, C₁₋₆ alkyl, C₁₋₆ haloalkyl, halo, or CN;
Cy² is an optionally substituted group selected from phenyl, pyridyl, or a 4-7 membered heterocycloalkyl ring having 1-2 heteroatoms independently selected from nitrogen, oxygen, or sulfur;
each R^{3a}, and R^{3b} is independently H or C₁₋₆ alkyl;
each R^{4a} and R^{4b} is independently H, halo, CN, OR, —N(R)₂, —C(O)N(R)₂, —NRC(O)R, —SO₂R, —C(O)R, —CO₂R, or an optionally substituted group selected from C₁₋₆ alkyl, C₃₋₇ cycloalkyl, a 4-7 membered heterocycloalkyl ring having 1-2 heteroatoms independently selected from nitrogen, oxygen, or sulfur, phenyl, an 8-10 membered bicyclic aryl ring, and a 5-6 membered heteroaryl ring having 1-4 heteroatoms independently selected from nitrogen, oxygen, or sulfur;
each R is independently H, or an optionally substituted group selected from C₁₋₆ aliphatic, phenyl, an 8-10 membered bicyclic aryl ring, a 4-7 membered saturated or partially unsaturated heterocyclic ring having 1-2 heteroatoms independently selected from nitrogen, oxygen, or sulfur, and a 5-6 membered heteroaryl ring having 1-4 heteroatoms independently selected from nitrogen, oxygen, or sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, or sulfur;
R^{5a} is H, C₁₋₆ alkyl, C₁₋₆ haloalkyl, halo, or CN;
each R^{6a} and R^{6b} is independently H or C₁₋₆ alkyl; or R^{6a} and R^{6b} are joined together to form a bond;
R^{6c} is H or substituted or unsubstituted C₁₋₆ alkyl;
m is 1, 2, or 3; and n is 1, 2, 3, or 4.

In some embodiments, W is —S(O)—, or —S(O)₂—.

In some embodiments, W is —C(O)—.

In some embodiments, X is —NR^{3a}—; and Y is —C(R^{3b})₂—, —NR^{3b}— or —O—.

In some embodiments, Y is a single bond, or —NR^{3a}—; and X is —C(R^{3b})₂—, —NR^{3b}—, or —O—.

In some embodiments, each of X and Y is independently —NR^{3a}—.

In some embodiments, R^{3a} is H.

In some embodiments, R^{3b} is H or Me.

In some embodiments, each of X and Y is —N(H)—.

In some embodiments, —X—W—Y— is —N(H)—C(O)—N(H)—, —N(H)—C(O)—CH₂—, —CH₂—C(O)—N(H)—, —N(H)—S(O)—N(H)—, —N(H)—S(O)—CH₂—, —CH₂—S(O)—N(H)—, —N(H)—S(O)₂—N(H)—, —N(H)—S(O)₂—CH₂—, —CH₂—S(O)₂—N(H)—, or —N(H)—C(O)—.

In some embodiments, R¹ is Cy²-N(H)C(O)—C(R^{6a})═C(R^{6b})(R^{6c}), or CH₂—Cy²-N(H)C(O)—C(R^{6a})═C(R^{6b})(R^{6c}); and R² is H, halo, hydroxyl, CN, substituted or unsubstituted C₁₋₆alkyl, substituted or unsubstituted amino, or substituted or unsubstituted alkoxy.

In some embodiments, R¹ is Cy²-N(H)C(O)—C(R^{6a})═C(R^{6b})(R^{6c}), or CH₂—Cy²-N(H)C(O)—C(R^{6a})═C(R^{6b})(R^{6c}); and R² is H, Me, Et, i-Pr, CF₃, F, Cl, OMe, OEt, or CN.

In some embodiments, R¹ is Cy²-N(H)C(O)—C(R^{6a})═C(R^{6b})(R^{6c}), or CH₂—Cy²-N(H)C(O)—C(R^{6a})═C(R^{6b})(R^{6c}); and R² is H.

In some embodiments, R² is Cy²-N(H)C(O)—C(R^{6a})═C(R^{6b})(R^{6c}), or CH₂-Cy²-N(H)C(O)—C(R^{6a})═C(R^{6b})(R^{6c}); and R¹ is H, halo, hydroxyl, CN, substituted or unsubstituted C₁₋₆alkyl, substituted or unsubstituted amino, or substituted or unsubstituted alkoxy.

In some embodiments, R² is Cy²-N(H)C(O)—C(R^{6a})═C(R^{6b})(R^{6c}), or CH₂-Cy²-N(H)C(O)—C(R^{6a})═C(R^{6b})(R^{6c}); and R¹ is H, Me, Et, i-Pr, CF₃, F, Cl, OMe, OEt, or CN.

In some embodiments, R² is Cy²-N(H)C(O)—C(R^{6a})═C(R^{6b})(R^{6c}), or CH₂—Cy²-N(H)C(O)—C(R^{6a})═C(R^{6b})(R^{6c}); and R¹ is H.

The compound according claim 1, wherein —X—W—Y— is —N(H)—C(O)—; R¹ is —CH₂—Cy²-N(H)C(O)—C(R^{6a})═C(R^{6b})(R^{6c}); and R² is H.

In some embodiments, the compound is according to formula (XXI):

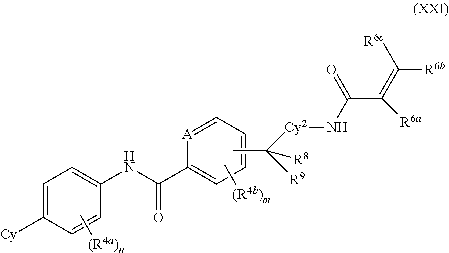

or a pharmaceutically acceptable salt thereof,
wherein A, Cy, Cy², R^{4b}, R^{6a}, R^{6b}, R^{6c}, m, and n are as described for formula (I); and each R⁸ and R⁹ is independently H, C₁₋₆ alkyl, C₁₋₆ haloalkyl, halo, or CN.

In some embodiments, one of R⁸ and R⁹ is H, halo, hydroxyl, CN, substituted or unsubstituted C₁₋₆alkyl, substituted or unsubstituted amino, or substituted or unsubstituted alkoxy; and the other is H.

In some embodiments, each R⁸ and R⁹ is H, or Me.

In some embodiments, each R⁸ and R⁹ is H.

In some embodiments, A is N.

In some embodiments, A is C.

In some embodiments, m is 1 or 2.

In some embodiments, n is 1 or 2.

In some embodiments, each R^{4a} is independently H, halo, hydroxyl, CN, substituted or unsubstituted C₁₋₆alkyl, substituted or unsubstituted amino, or substituted or unsubstituted alkoxy.

In some embodiments, each R^{4a} is independently H, Me, Et, i-Pr, CF₃, F, Cl, OMe, OEt, or CN.

In some embodiments, each R^{4a} is H.

In some embodiments, each R^{4b} is independently H, halo, hydroxyl, CN, substituted or unsubstituted C₁₋₆alkyl, substituted or unsubstituted amino, or substituted or unsubstituted alkoxy.

In some embodiments, each R^{4b} is independently H, Me, Et, i-Pr, CF₃, F, Cl, OMe, OEt, or CN.

In some embodiments, each R^{4b} is H.

In some embodiments, the compound is according to formula (IIa), (IIb), (IIc) or (IId):

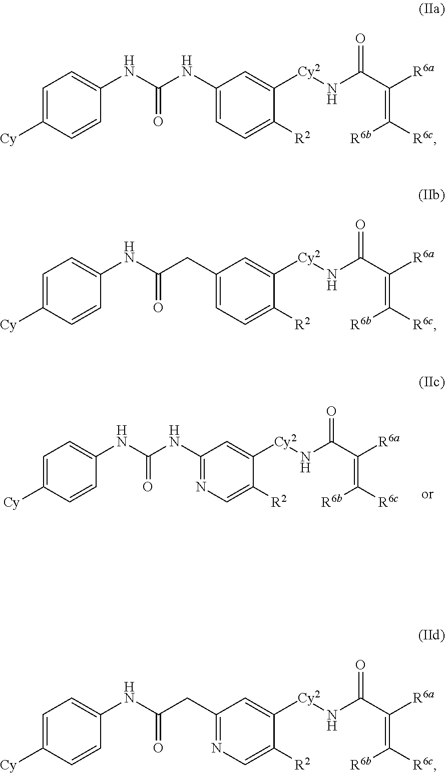

or a pharmaceutically acceptable salt thereof.

In some embodiments, R² is H, Me, Et, i-Pr, CF₃, F, Cl, OMe, OEt, or CN.

In some embodiments, R² is H.

In some embodiments, the compound is according to formula (XXIIa) or (XXIIb):

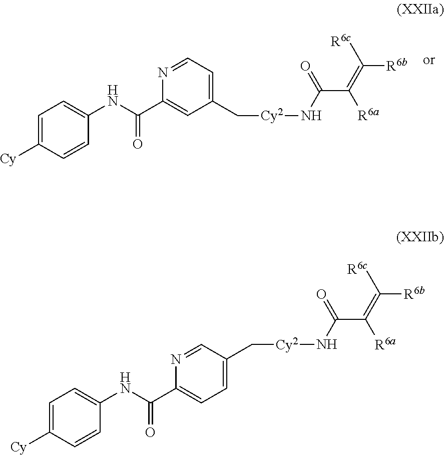

or a pharmaceutically acceptable salt thereof; wherein Cy, Cy², R^{6a}, R^{6b}, or R^{6c} are as described for formula (I).

In some embodiments, the compound is according to formula (IIIa), (IIIb), (IIIc) or (IIId):

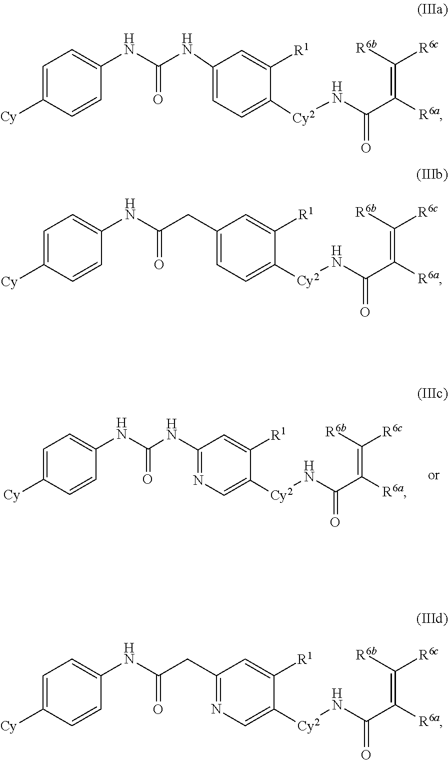

or a pharmaceutically acceptable salt thereof.

In some embodiments, The compound according to claim 1, wherein the compound is according to formula (XXXIIa), (XXXIIb), (XXXIIc), (XXXIId), (XXXIIe), or (XXXIIf):

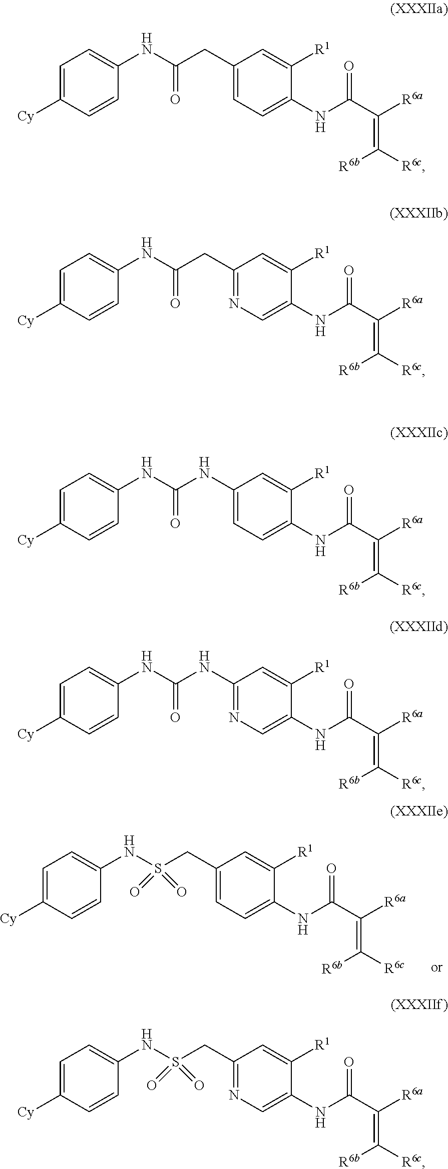

or a pharmaceutically acceptable salt thereof.

In some embodiments, R¹ is H, Me, Et, i-Pr, CF₃, F, Cl, OMe, OEt, or CN.

In some embodiments, R¹ is H.

In some embodiments, wherein the compound is according to formula (XXXIIIa), (XXXIIIb), (XXXIIIc), (XXXIIId), (XXXIIIe), or (XXXIIIf):

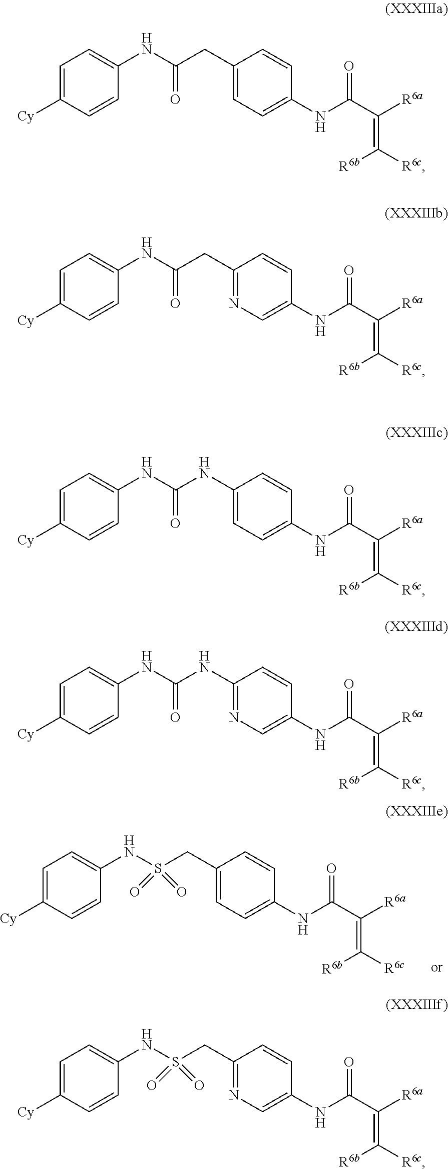

or a pharmaceutically acceptable salt thereof.

In some embodiments, Cy² is substituted or unsubstituted Ph, pyridyl, azetidinyl, pyrrolidinyl, piperidinyl, or azepinyl.

In some embodiments, the compound is according to formula (IVa), or (IVb):

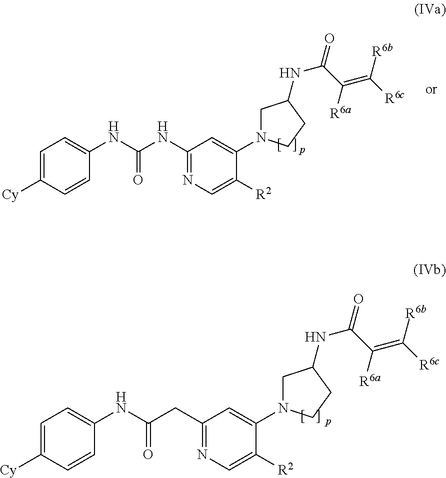

or a pharmaceutically acceptable salt thereof; and wherein p is 0, 1, 2, or 3.

In some embodiments, the compound is according to formula (XXIIIa) or (XXIIIb):

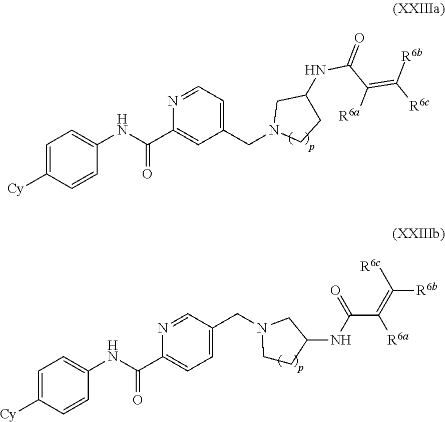

or a pharmaceutically acceptable salt thereof; and wherein p is 0, 1, 2, or 3.

In some embodiments, Cy is substituted or unsubstituted

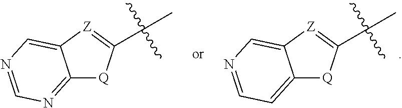

In some embodiments, Cy is substituted or unsubstituted

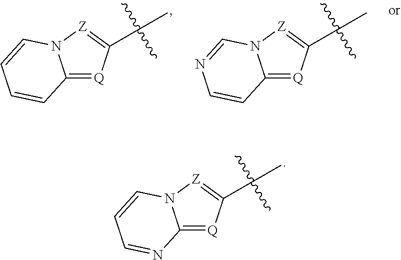

In some embodiments, Q is —N(H)—.

In some embodiments, Q is —O—.

In some embodiments, Q is —S—.

In some embodiments, Z is —N═.

In some embodiments, Z is —CR^{5a}═.

In some embodiments, R^{5a} is H, Me, Et, i-Pr, Cl, F, CF₃, or CN.

In some embodiments, R^{5a} is H, Me, or F.

In some embodiments, R^{5a} is H.

In some embodiments, Z is —C(H)═.

In some embodiments, Cy is

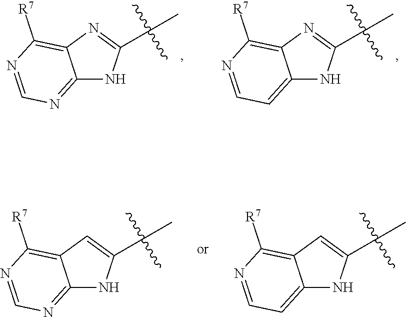

wherein R⁷ is an optionally substituted group selected from a 4-7 membered heterocycloalkyl ring having 1-2 heteroatoms independently selected from nitrogen, oxygen, or sulfur, phenyl, an 8-10 membered bicyclic aryl ring, and a 5-6 membered heteroaryl ring having 1-4 heteroatoms independently selected from nitrogen, oxygen, or sulfur.

In some embodiments, Cy is substituted or unsubstituted

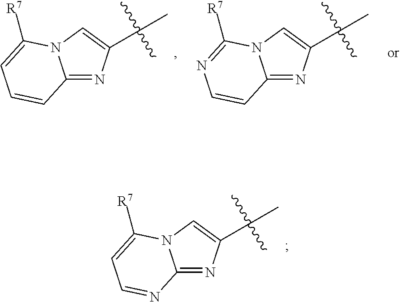

wherein R⁷ is an optionally substituted group selected from a 4-7 membered heterocycloalkyl ring having 1-2 heteroatoms independently selected from nitrogen, oxygen, or sulfur, phenyl, an 8-10 membered bicyclic aryl ring, and a 5-6 membered heteroaryl ring having 1-4 heteroatoms independently selected from nitrogen, oxygen, or sulfur.

In some embodiments, the compound is according to formula (Va), or (Vb).

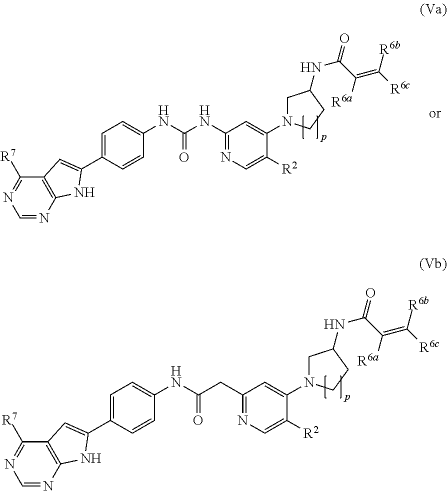

or a pharmaceutically acceptable salt thereof; and wherein p is 0, 1, 2, or 3; and R⁷ is an optionally substituted group selected from a 4-7 membered heterocycloalkyl ring having 1-2 heteroatoms independently selected from nitrogen, oxygen, or sulfur, phenyl, an 8-10 membered bicyclic aryl ring, and a 5-6 membered heteroaryl ring having 1-4 heteroatoms independently selected from nitrogen, oxygen, or sulfur.

In some embodiments, the compound is according to formula (XXIVa), or (XXIVb):

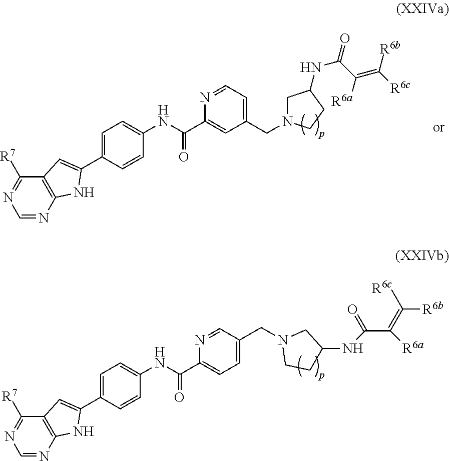

or a pharmaceutically acceptable salt thereof; and wherein p is 0, 1, 2, or 3; and R⁷ is an optionally substituted group selected from a 4-7 membered heterocycloalkyl ring having 1-2 heteroatoms independently selected from nitrogen, oxygen, or sulfur, phenyl, an 8-10 membered bicyclic aryl ring, and a 5-6 membered heteroaryl ring having 1-4 heteroatoms independently selected from nitrogen, oxygen, or sulfur.

In some embodiments, the compound is according to formula (XXXIVa), or (XXXIVb):

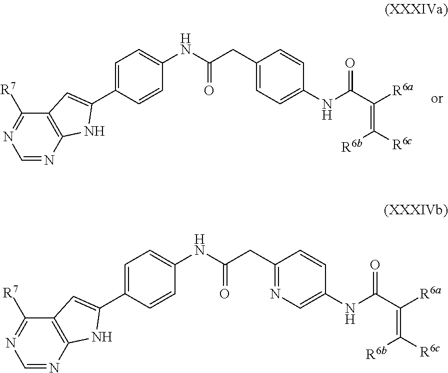

or a pharmaceutically acceptable salt thereof.

In some embodiments, the compound is according to formula (XXXVa), or (XXXVb):

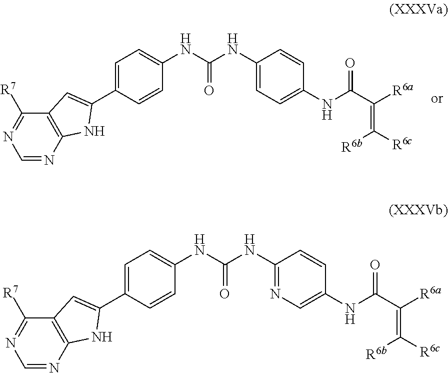

or a pharmaceutically acceptable salt thereof.

In some embodiments, the compound is according to formula (XXXVIa), or (XXXVIb):

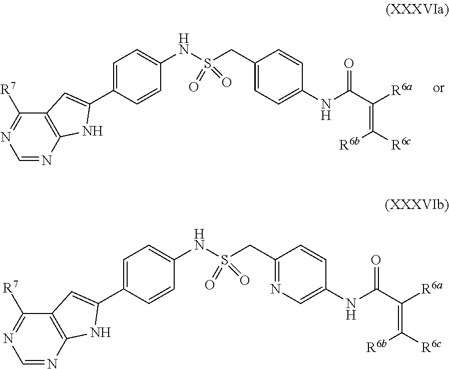

or a pharmaceutically acceptable salt thereof.

In some embodiments, R⁷ is 4-7 membered heterocycloalkyl ring having 1-2 heteroatoms independently selected from nitrogen, oxygen, or sulfur substituted with Me, Et, or i-Pr.

In some embodiments, R⁷ is pyrrolidinyl, piperidinyl, piperazinyl, or morpholinyl.

In some embodiments, R⁷ is morpholinyl.

In some embodiments, R⁷ is substituted or unsubstituted heteroaryl.

In some embodiments, R⁷ is substituted or unsubstituted pyridyl or pyrimidyl.

In some embodiments, R⁷ is unsubstituted pyridyl.

In some embodiments, R⁷ is pyridyl substituted with halo, hydroxyl, CN, substituted or unsubstituted C₁₋₆alkyl, substituted or unsubstituted amino, or substituted or unsubstituted alkoxy.

In some embodiments, R⁷ is pyridyl substituted with Me, Et, i-Pr, OH, Cl, F, CF₃, CN, or NH₂.

In some embodiments, R⁷ is pyridyl substituted with Me, Et, i-Pr, Cl, F, CF₃, or CN.

In some embodiments, R⁷ is substituted or unsubstituted pyrrolyl, pyrazolyl, imidazolyl, oxazolyl, triazolyl, thiazolyl, oxadiazolyl, or thiadiazolyl.

In some embodiments, R⁷ is substituted or unsubstituted imidazolyl.

In some embodiments, R⁷ is imidazoyl substituted with Me, Et, i-Pr, Cl, F, CF₃, or CN.

In some embodiments, R⁷ is imidazoyl substituted with Me.

In some embodiments, the compound is according to formula (VIa), or (VIb):

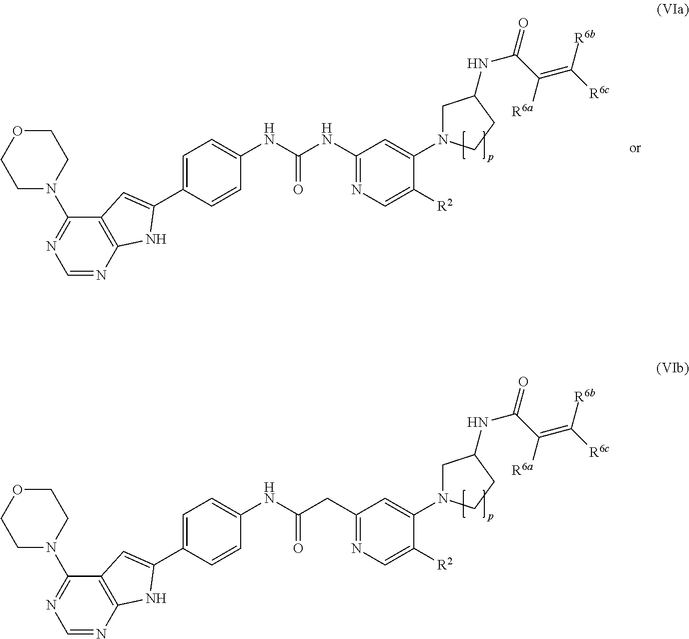

or a pharmaceutically acceptable salt thereof; and wherein p is 0, 1, 2, or 3.

In some embodiments, the compound is according to formula (XXVa), or (XXVb):

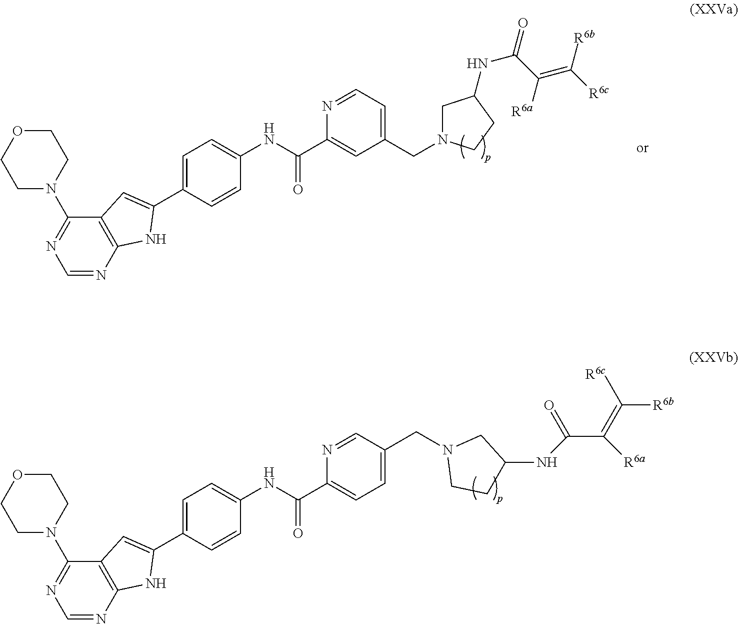

or a pharmaceutically acceptable salt thereof; and wherein p is 0, 1, 2, or 3.

In some embodiments, p is 0, 1, or 2.

In some embodiments, R² is H or F.

In some embodiments, R² is H.

In some embodiments, the compound is according to formula (VIIa), (VIIb), or (VIIc):

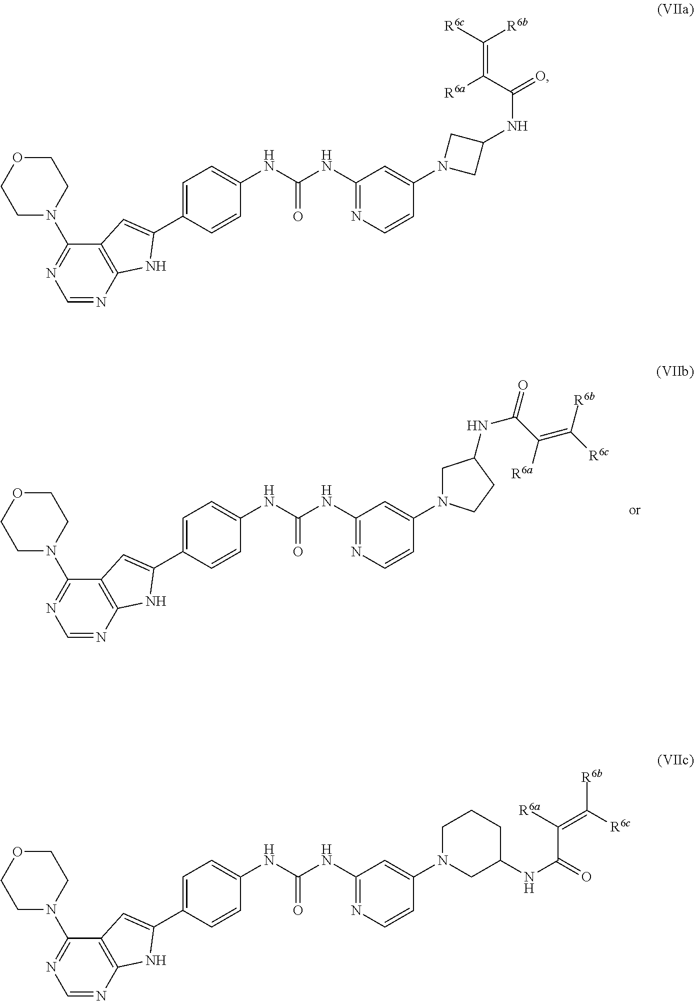

or a pharmaceutically acceptable salt thereof.

In some embodiments, the compound is according to formula (VIIIa), (VIIIb), or (VIIIc):

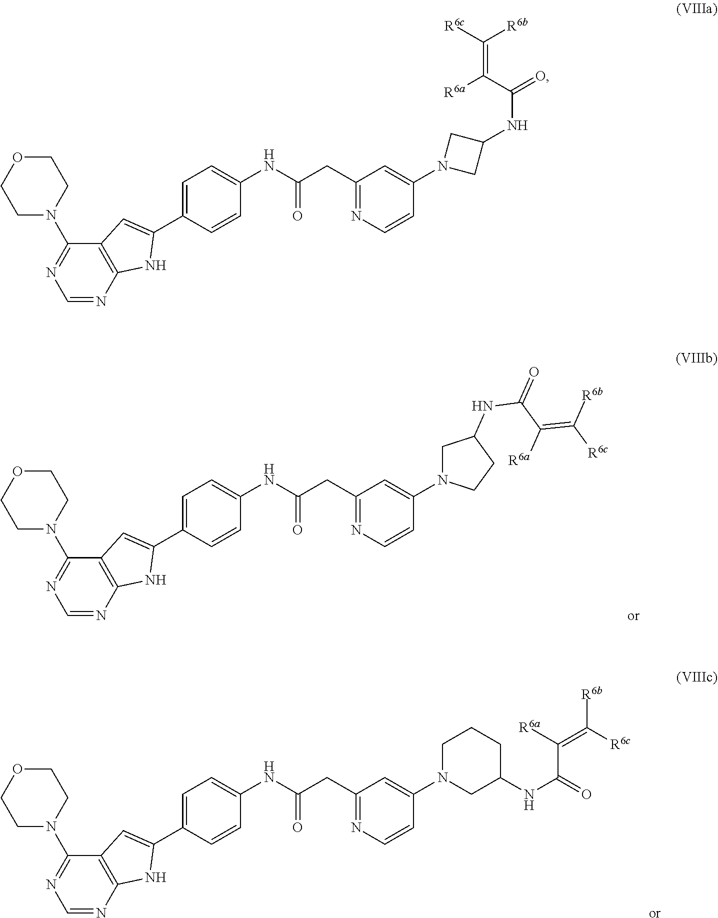

or a pharmaceutically acceptable salt thereof.

In some embodiments, the compound is according to formula (XXVIa), (XXVIb), or (XXVIc).

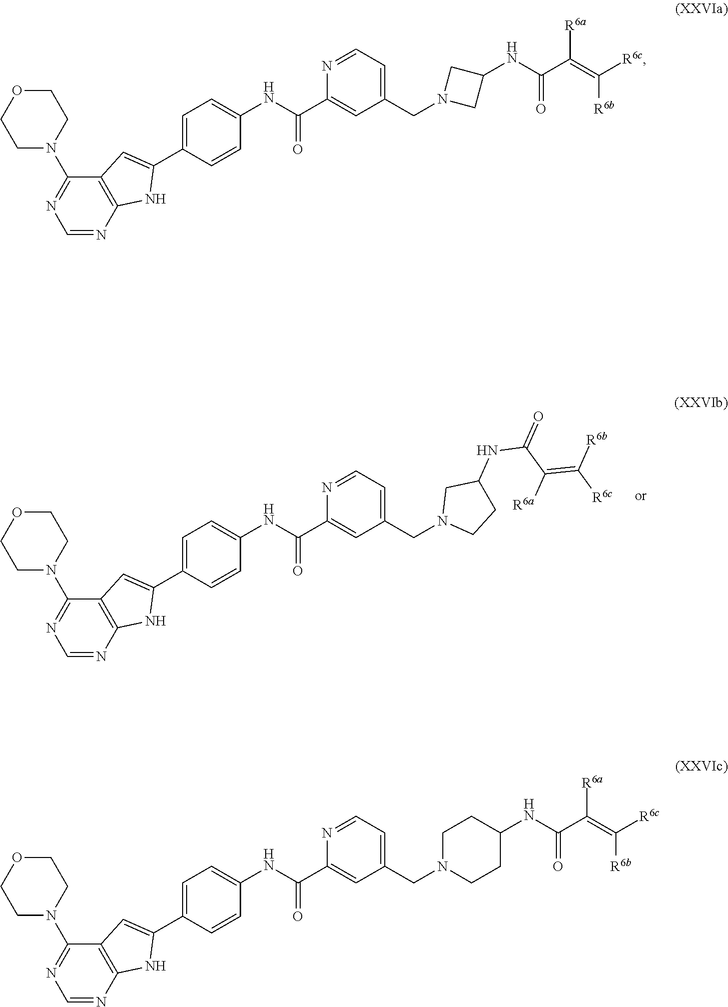

or a pharmaceutically acceptable salt thereof.

In some embodiments, the compound is according to formula (XXXVIIa), or (XXXVIIb):

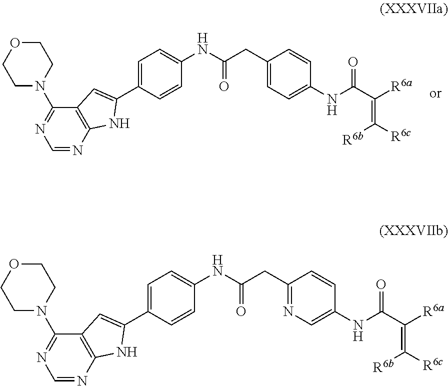

or a pharmaceutically acceptable salt thereof.

In some embodiments, the compound is according to formula (XXXVIIIa), or (XXXVIIIb):

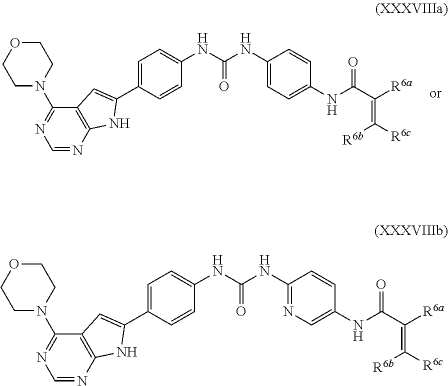

or a pharmaceutically acceptable salt thereof.

In some embodiments, the compound is according to formula (XXXIXa), or (XXXIXb):

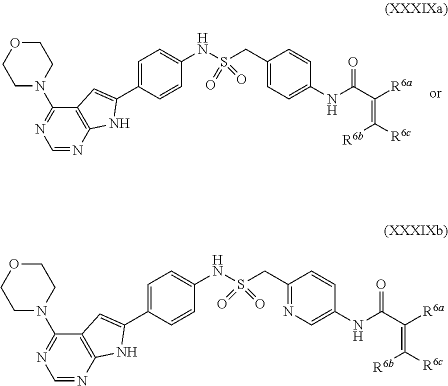

or a pharmaceutically acceptable salt thereof.

In some embodiments, each of R^{6a}, R^{6b}, and R^{6c} is H.

In some embodiments, each of R^{6a}, and R^{6b} is H; and R^{6c} is substituted or unsubstituted alkyl.

In some embodiments, each of R^{6a}, and R^{6b} is H; and R^{6c} is unsubstituted alkyl.

In some embodiments, each of R^{6a}, and R^{6b} is H; and R^{6c} is Me, or Et.

In some embodiments, each of R^{6a}, and R^{6b} is H; and R^{6c} is alkyl substituted with amino, alkylamino or dialkylamino.

In some embodiments, each of R^{6a}, and R^{6b} is H; and R^{6c} is alkyl substituted with dimethylamino.

In some embodiments, each of R^{6a}, and R^{6b} is H; and R^{6c} is —CH₂NMe₂.

In some embodiments, R^{6a}, and R^{6b} form a bond; and R^{6c} is H or substituted or unsubstituted alkyl.

In some embodiments, R^{6a}, and R^{6b} form a bond; and R^{6c} is Me.

In some embodiments, the compound is according to formula (IXa), (IXb), or (IXc):

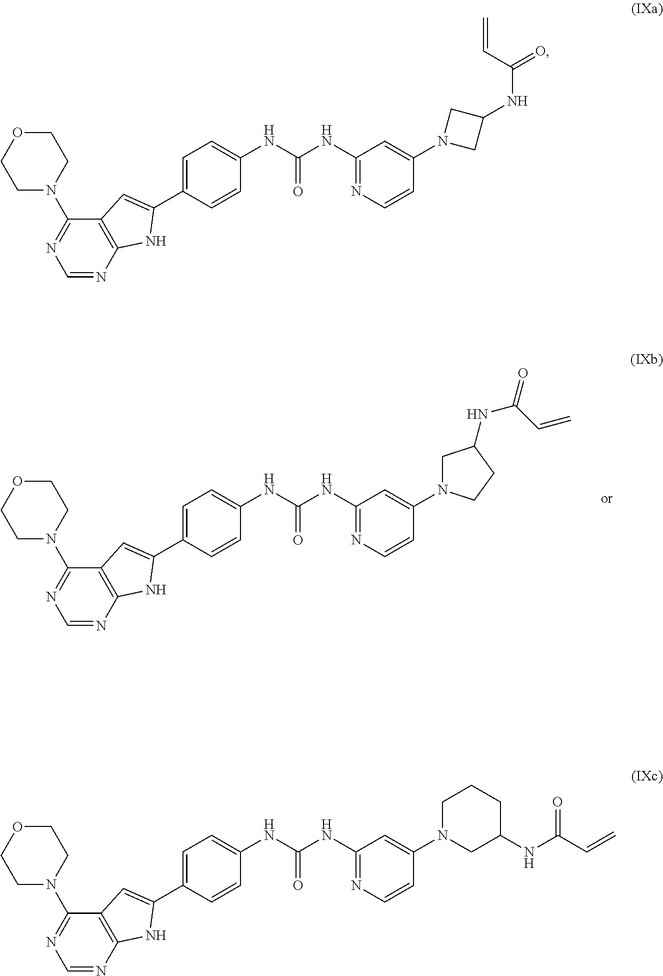 [SMILES: C=CC(=O)NC1CN(c2ccnc(NC(=O)Nc3ccc(-c4cc5c(N6CCOCC6)ncnc5[nH]4)cc3)c2)C1 ; C=CC(=O)NC1CCN(c2ccnc(NC(=O)Nc3ccc(-c4cc5c(N6CCOCC6)ncnc5[nH]4)cc3)c2)C1 ; C=CC(=O)NC1CCCN(c2ccnc(NC(=O)Nc3ccc(-c4cc5c(N6CCOCC6)ncnc5[nH]4)cc3)c2)C1]

or a pharmaceutically acceptable salt thereof.

In some embodiments, the compound is according to formula (Xa), (Xb), or (Xc):

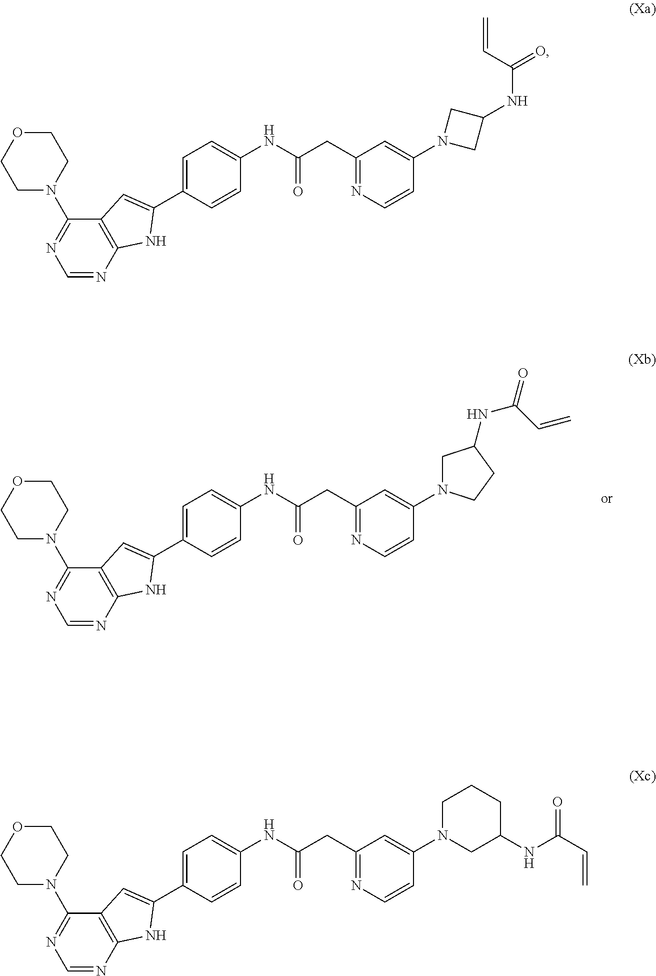 [SMILES: C=CC(=O)NC1CN(c2ccnc(CC(=O)Nc3ccc(-c4cc5c(N6CCOCC6)ncnc5[nH]4)cc3)c2)C1 ; C=CC(=O)NC1CCN(c2ccnc(CC(=O)Nc3ccc(-c4cc5c(N6CCOCC6)ncnc5[nH]4)cc3)c2)C1 ; C=CC(=O)NC1CCCN(c2ccnc(CC(=O)Nc3ccc(-c4cc5c(N6CCOCC6)ncnc5[nH]4)cc3)c2)C1]

or a pharmaceutically acceptable salt thereof.

In some embodiments, the compound is according to formula (XIa), (XIb), or (XIc):

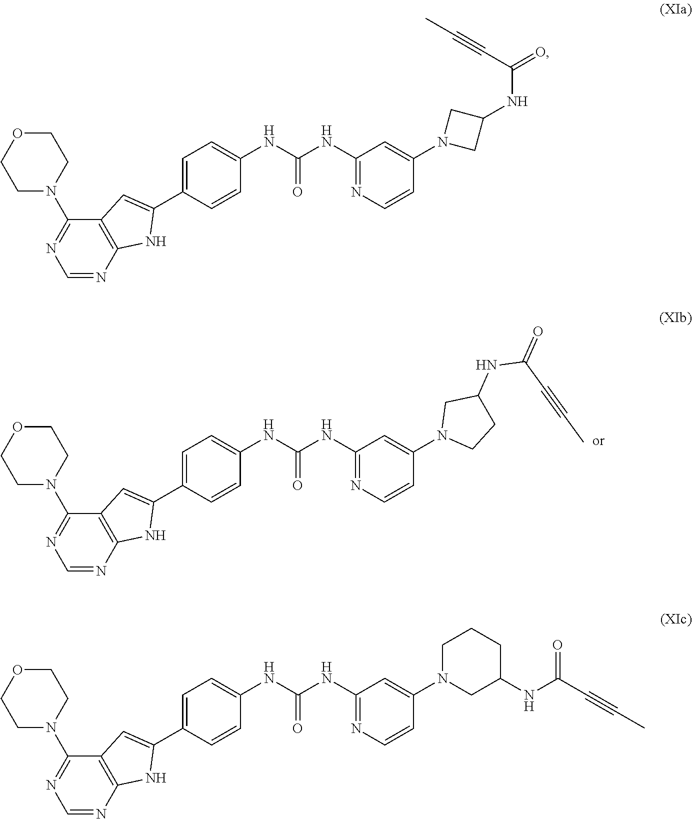 [SMILES: CC#CC(=O)NC1CN(c2ccnc(NC(=O)Nc3ccc(-c4cc5c(N6CCOCC6)ncnc5[nH]4)cc3)c2)C1 ; CC#CC(=O)NC1CCN(c2ccnc(NC(=O)Nc3ccc(-c4cc5c(N6CCOCC6)ncnc5[nH]4)cc3)c2)C1 ; CC#CC(=O)NC1CCCN(c2ccnc(NC(=O)Nc3ccc(-c4cc5c(N6CCOCC6)ncnc5[nH]4)cc3)c2)C1]

or a pharmaceutically acceptable salt thereof.

In some embodiments, the compound is according to formula (XIIa), (XIIb), or (XIIc):

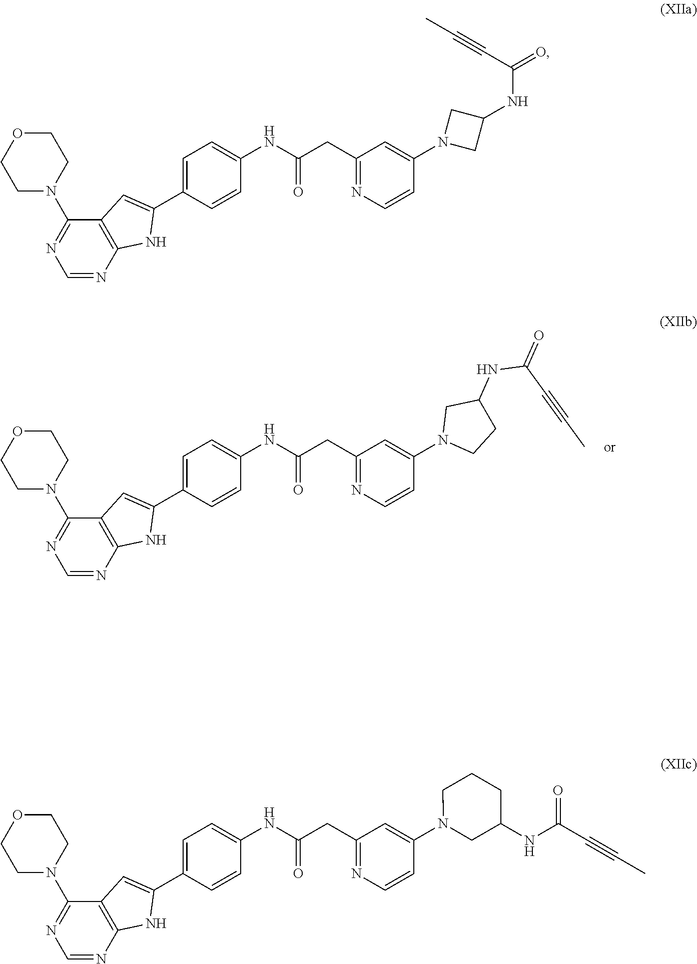 [SMILES: CC#CC(=O)NC1CN(c2ccnc(CC(=O)Nc3ccc(-c4cc5c(N6CCOCC6)ncnc5[nH]4)cc3)c2)C1 ; CC#CC(=O)NC1CCN(c2ccnc(CC(=O)Nc3ccc(-c4cc5c(N6CCOCC6)ncnc5[nH]4)cc3)c2)C1 ; CC#CC(=O)NC1CCCN(c2ccnc(CC(=O)Nc3ccc(-c4cc5c(N6CCOCC6)ncnc5[nH]4)cc3)c2)C1]

or a pharmaceutically acceptable salt thereof.

In some embodiments, the compound is according to formula (XIIIa), (XIIIb), or (XIIIc):

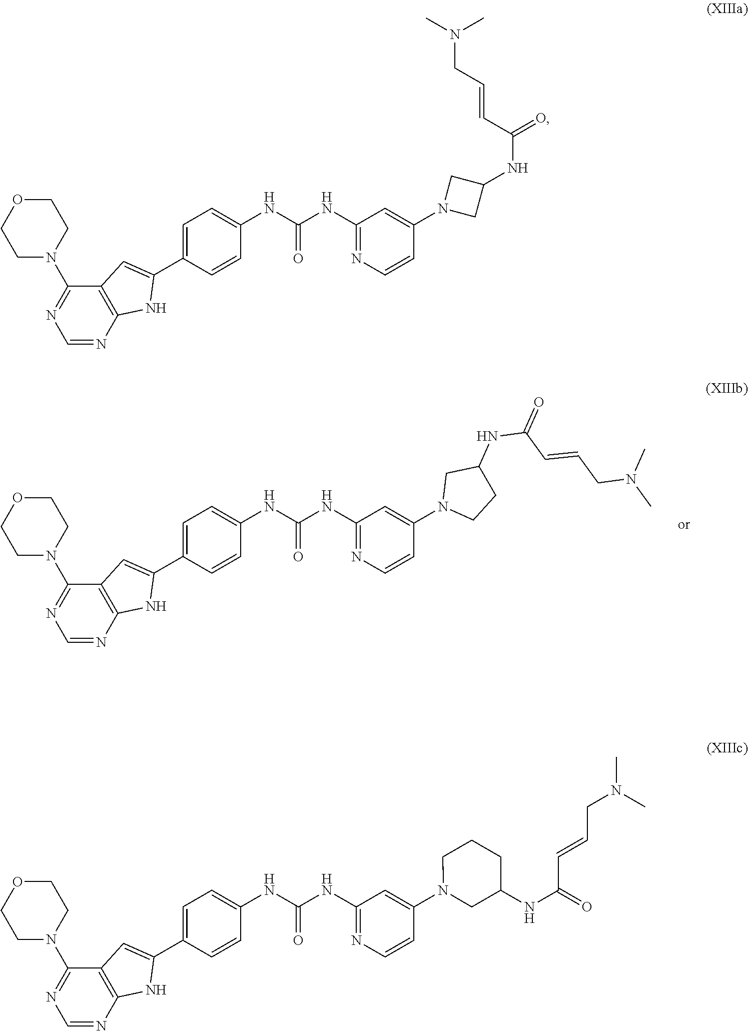 [SMILES: CN(C)C/C=C/C(=O)NC1CN(c2ccnc(NC(=O)Nc3ccc(-c4cc5c(N6CCOCC6)ncnc5[nH]4)cc3)c2)C1 ; CN(C)C/C=C/C(=O)NC1CCN(c2ccnc(NC(=O)Nc3ccc(-c4cc5c(N6CCOCC6)ncnc5[nH]4)cc3)c2)C1 ; CN(C)C/C=C/C(=O)NC1CCCN(c2ccnc(NC(=O)Nc3ccc(-c4cc5c(N6CCOCC6)ncnc5[nH]4)cc3)c2)C1]

or a pharmaceutically acceptable salt thereof.

In some embodiments, the compound is according to formula (XIVa), (XIVb), or (XIVc):

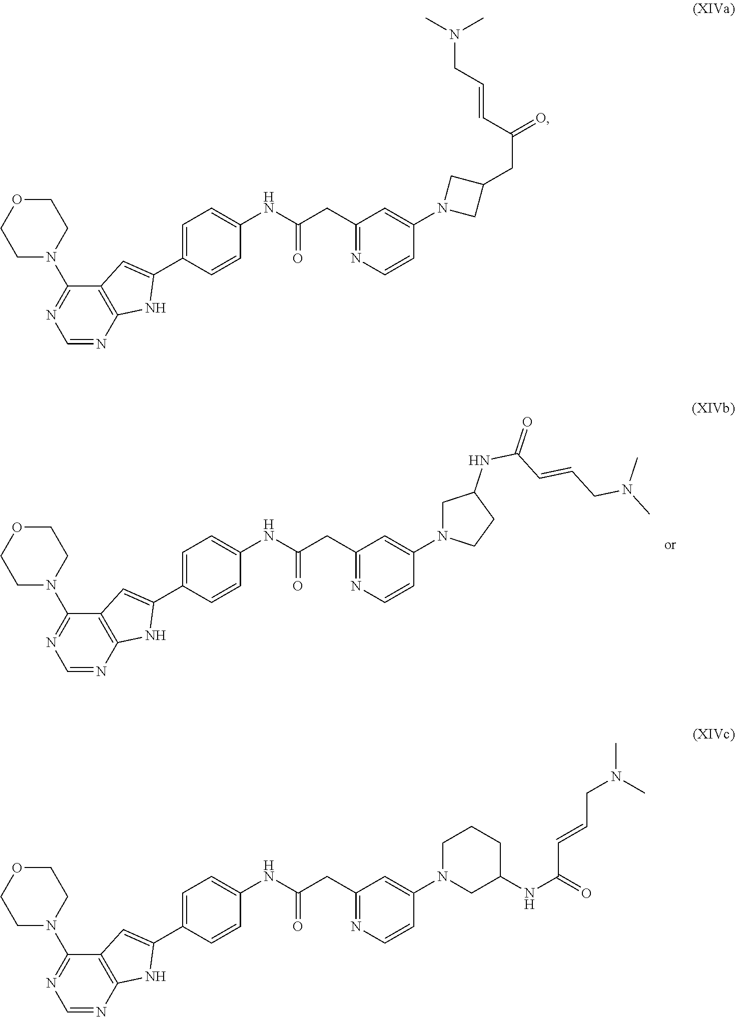 [SMILES: CN(C)C/C=C/C(=O)CC1CN(c2ccnc(CC(=O)Nc3ccc(-c4cc5c(N6CCOCC6)ncnc5[nH]4)cc3)c2)C1 ; CN(C)C/C=C/C(=O)NC1CCN(c2ccnc(CC(=O)Nc3ccc(-c4cc5c(N6CCOCC6)ncnc5[nH]4)cc3)c2)C1 ; CN(C)C/C=C/C(=O)NC1CCCN(c2ccnc(CC(=O)Nc3ccc(-c4cc5c(N6CCOCC6)ncnc5[nH]4)cc3)c2)C1]

or a pharmaceutically acceptable salt thereof.

In some embodiments, the compound is according to formula (XV):

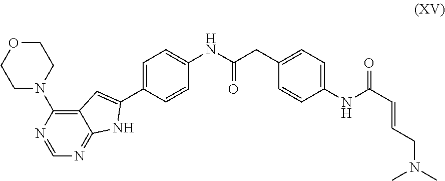 [SMILES: CN(C)C/C=C/C(=O)Nc1ccc(CC(=O)Nc2ccc(-c3cc4c(N5CCOCC5)ncnc4[nH]3)cc2)cc1]

or a pharmaceutically acceptable salt thereof.

In some embodiments, the compound is according to formula (XVI):

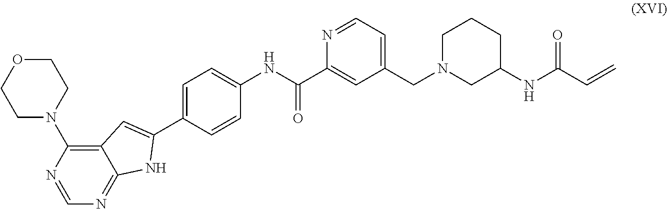 [SMILES: C=CC(=O)NC1CCCN(Cc2ccnc(C(=O)Nc3ccc(-c4cc5c(N6CCOCC6)ncnc5[nH]4)cc3)c2)C1]

or a pharmaceutically acceptable salt thereof.

In some embodiments, the compound is according to formula (XVII):

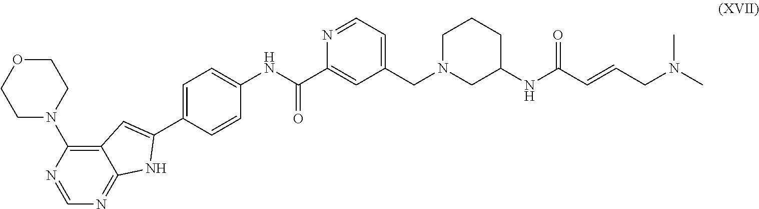 [SMILES: CN(C)C/C=C/C(=O)NC1CCCN(Cc2ccnc(C(=O)Nc3ccc(-c4cc5c(N6CCOCC6)ncnc5[nH]4)cc3)c2)C1]

or a pharmaceutically acceptable salt thereof.

In some embodiments, the compound is according to formula (XXVIIa), (XXVIIb), or (XXVIIc):

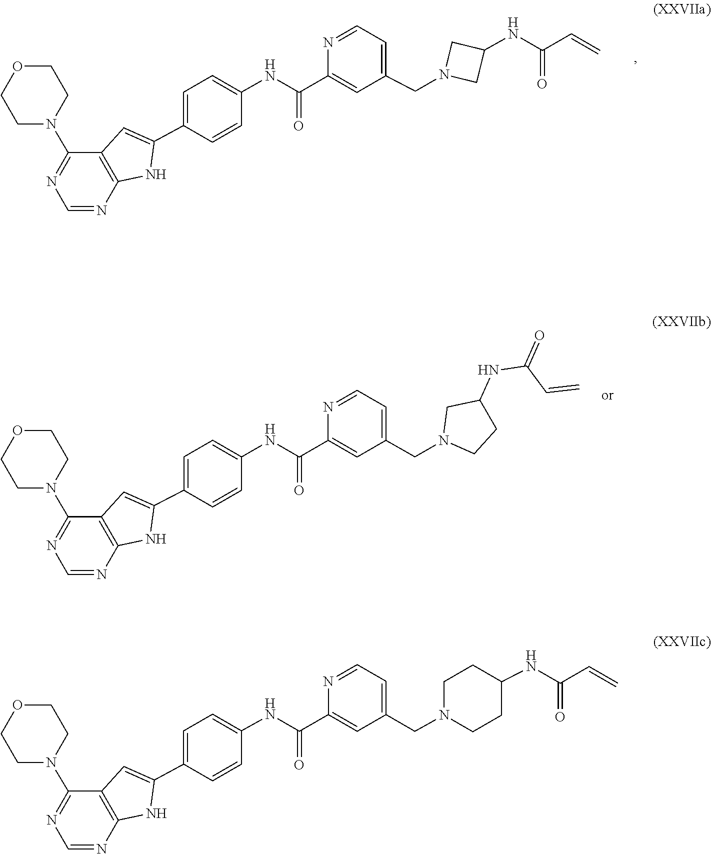 [SMILES: C=CC(=O)NC1CN(Cc2ccnc(C(=O)Nc3ccc(-c4cc5c(N6CCOCC6)ncnc5[nH]4)cc3)c2)C1 ; C=CC(=O)NC1CCN(Cc2ccnc(C(=O)Nc3ccc(-c4cc5c(N6CCOCC6)ncnc5[nH]4)cc3)c2)C1 ; C=CC(=O)NC1CCN(Cc2ccnc(C(=O)Nc3ccc(-c4cc5c(N6CCOCC6)ncnc5[nH]4)cc3)c2)CC1]

or a pharmaceutically acceptable salt thereof.

In some embodiments, the compound is according to formula (XXVIIIa), (XXVIIIb), or (XXVIIIc):

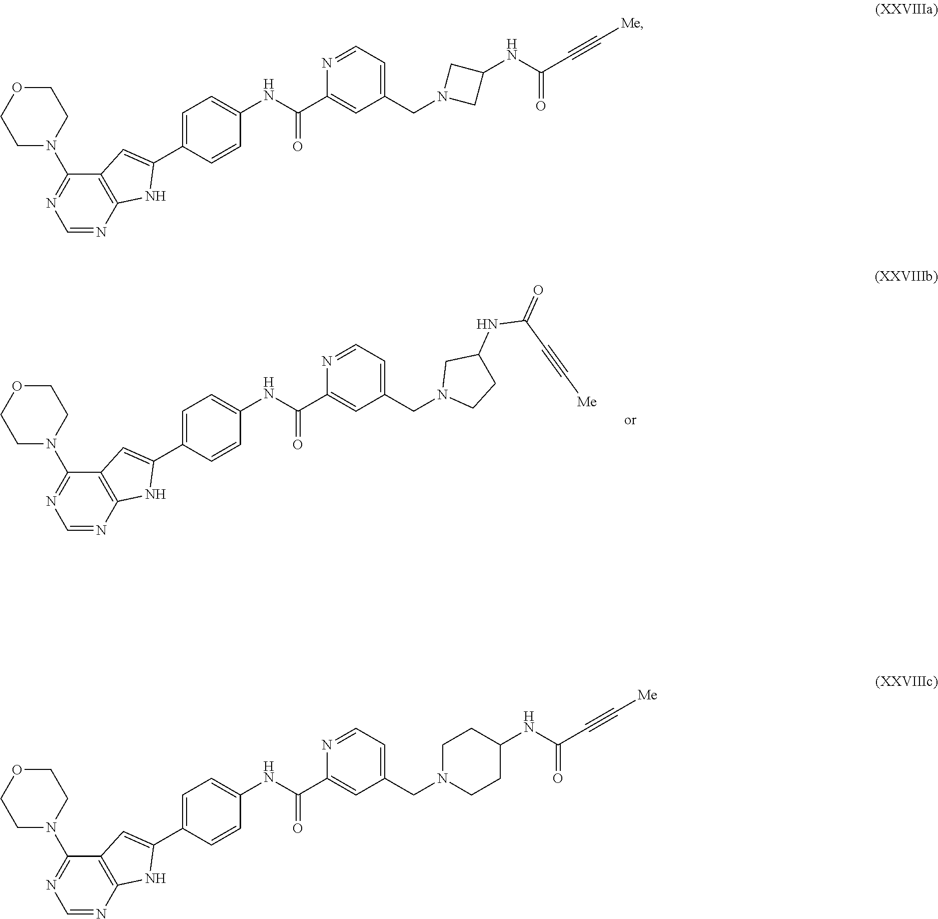 [SMILES: CC#CC(=O)NC1CN(Cc2ccnc(C(=O)Nc3ccc(-c4cc5c(N6CCOCC6)ncnc5[nH]4)cc3)c2)C1 ; CC#CC(=O)NC1CCN(Cc2ccnc(C(=O)Nc3ccc(-c4cc5c(N6CCOCC6)ncnc5[nH]4)cc3)c2)C1 ; CC#CC(=O)NC1CCN(Cc2ccnc(C(=O)Nc3ccc(-c4cc5c(N6CCOCC6)ncnc5[nH]4)cc3)c2)CC1]

or a pharmaceutically acceptable salt thereof.

In some embodiments, the compound is according to formula (XXIXa), (XXIXb), or (XXIXc):

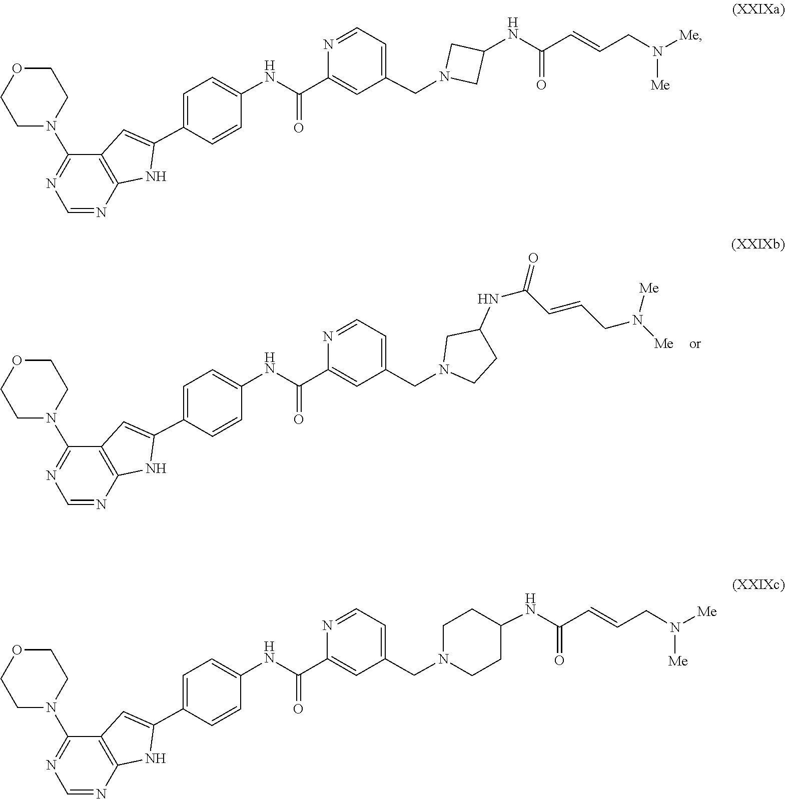 [SMILES: CN(C)C/C=C/C(=O)NC1CN(Cc2ccnc(C(=O)Nc3ccc(-c4cc5c(N6CCOCC6)ncnc5[nH]4)cc3)c2)C1 ; CN(C)C/C=C/C(=O)NC1CCN(Cc2ccnc(C(=O)Nc3ccc(-c4cc5c(N6CCOCC6)ncnc5[nH]4)cc3)c2)C1 ; CN(C)C/C=C/C(=O)NC1CCN(Cc2ccnc(C(=O)Nc3ccc(-c4cc5c(N6CCOCC6)ncnc5[nH]4)cc3)c2)CC1]

or a pharmaceutically acceptable salt thereof.

In some embodiments, the compound is according to formula (XLa), (XLb), or (XLc):

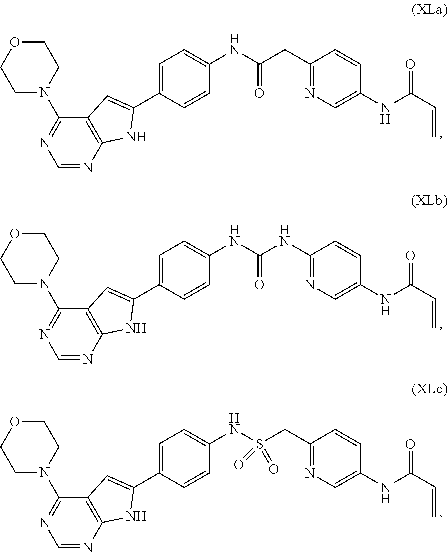 [SMILES: C=CC(=O)Nc1ccc(CC(=O)Nc2ccc(-c3cc4c(N5CCOCC5)ncnc4[nH]3)cc2)nc1 ; C=CC(=O)Nc1ccc(NC(=O)Nc2ccc(-c3cc4c(N5CCOCC5)ncnc4[nH]3)cc2)nc1 ; C=CC(=O)Nc1ccc(CS(=O)(=O)Nc2ccc(-c3cc4c(N5CCOCC5)ncnc4[nH]3)cc2)nc1]

or a pharmaceutically acceptable salt thereof.

In some embodiments, the compound is according to formula (XLIa), (XLIb), or (XLIc):

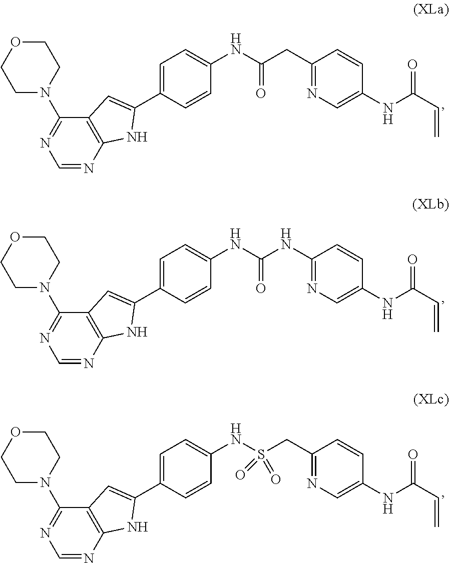 [SMILES: C=CC(=O)Nc1ccc(CC(=O)Nc2ccc(-c3cc4c(N5CCOCC5)ncnc4[nH]3)cc2)nc1 ; C=CC(=O)Nc1ccc(NC(=O)Nc2ccc(-c3cc4c(N5CCOCC5)ncnc4[nH]3)cc2)nc1 ; C=CC(=O)Nc1ccc(CS(=O)(=O)Nc2ccc(-c3cc4c(N5CCOCC5)ncnc4[nH]3)cc2)nc1]

or a pharmaceutically acceptable salt thereof.

In some embodiments, the compound is according to formula (XLIa), (XLIb), or (XLIc):

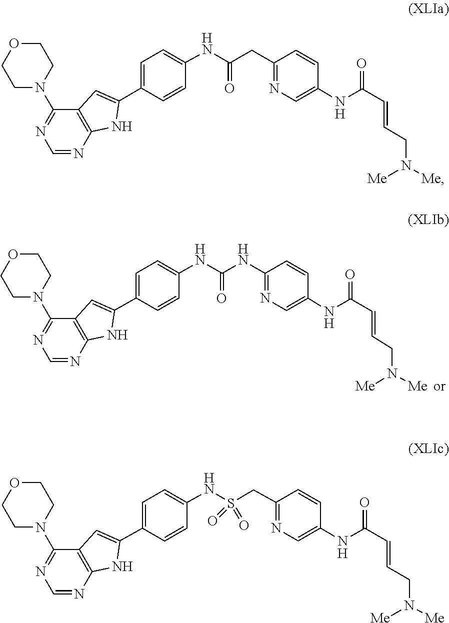 [SMILES: CN(C)C/C=C/C(=O)Nc1ccc(CC(=O)Nc2ccc(-c3cc4c(N5CCOCC5)ncnc4[nH]3)cc2)nc1 ; CN(C)C/C=C/C(=O)Nc1ccc(NC(=O)Nc2ccc(-c3cc4c(N5CCOCC5)ncnc4[nH]3)cc2)nc1 ; CN(C)C/C=C/C(=O)Nc1ccc(CS(=O)(=O)Nc2ccc(-c3cc4c(N5CCOCC5)ncnc4[nH]3)cc2)nc1]

or a pharmaceutically acceptable salt thereof.

In some embodiments, the compound is according to formula (XLIIa), (XLIIb), or (XLIIc):

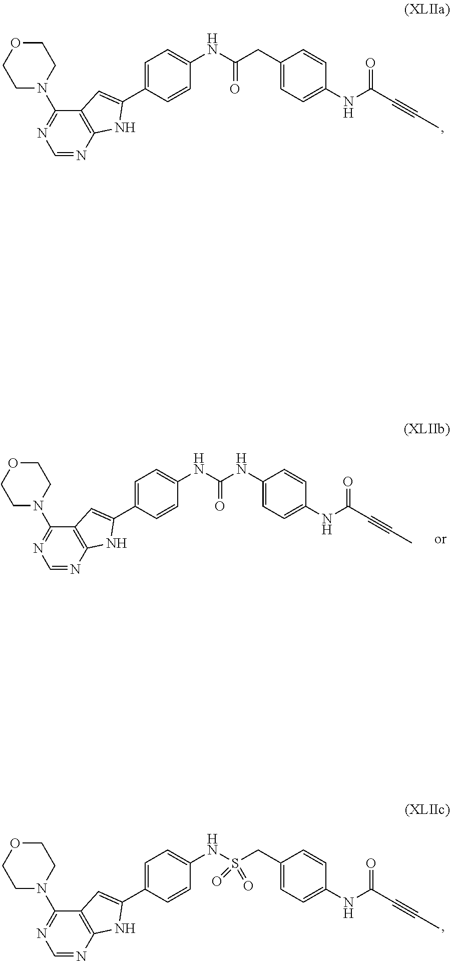 [SMILES: CC#CC(=O)Nc1ccc(CC(=O)Nc2ccc(-c3cc4c(N5CCOCC5)ncnc4[nH]3)cc2)cc1 ; CC#CC(=O)Nc1ccc(NC(=O)Nc2ccc(-c3cc4c(N5CCOCC5)ncnc4[nH]3)cc2)cc1 ; CC#CC(=O)Nc1ccc(CS(=O)(=O)Nc2ccc(-c3cc4c(N5CCOCC5)ncnc4[nH]3)cc2)cc1]

or a pharmaceutically acceptable salt thereof.

In some embodiments, the compound is according to formula (XLIIIa), (XLIIIb), or (XLIIIc):

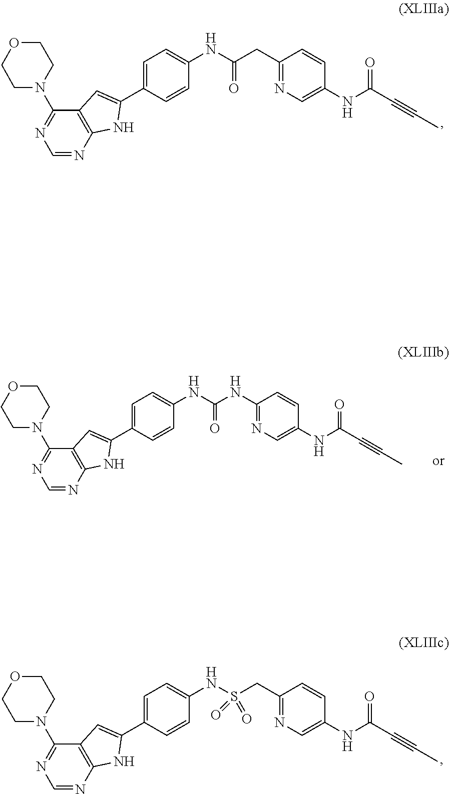 [SMILES: CC#CC(=O)Nc1ccc(CC(=O)Nc2ccc(-c3cc4c(N5CCOCC5)ncnc4[nH]3)cc2)nc1 ; CC#CC(=O)Nc1ccc(NC(=O)Nc2ccc(-c3cc4c(N5CCOCC5)ncnc4[nH]3)cc2)nc1 ; CC#CC(=O)Nc1ccc(CS(=O)(=O)Nc2ccc(-c3cc4c(N5CCOCC5)ncnc4[nH]3)cc2)nc1]

or a pharmaceutically acceptable salt thereof.

In certain embodiments, the compound is selected from compounds 1-26 and 101-112, provided herein. In certain embodiments, the compound is compound 10. In certain embodiments, the compound is the racemate of compound 10:

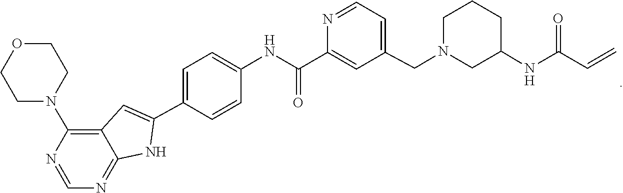 [SMILES: C=CC(=O)NC1CCCN(Cc2ccnc(C(=O)Nc3ccc(-c4cc5c(N6CCOCC6)ncnc5[nH]4)cc3)c2)C1]

In one particular embodiment, the compound is the R-isomer of compound 10:

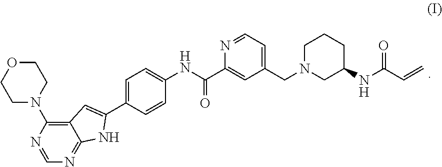 [SMILES: C=CC(=O)N[C@@H]1CCCN(Cc2ccnc(C(=O)Nc3ccc(-c4cc5c(N6CCOCC6)ncnc5[nH]4)cc3)c2)C1]

In one particular embodiment, the compound is N-[4-[4-(4-morpholinyl)-7H-pyrrolo[2,3-d]pyrimidin-6-yl]phenyl]-4-[[3(R)-[(1-oxo-2-propen-1-yl)amino]-1-piperidinyl]methyl]-2-pyridinecarboxamide, or a pharmaceutically acceptable salt thereof. In one particular embodiment, the compound is the S-isomer of compound 10:

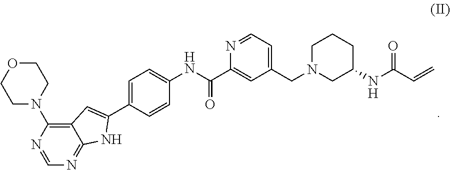 [SMILES: C=CC(=O)N[C@H]1CCCN(Cc2ccnc(C(=O)Nc3ccc(-c4cc5c(N6CCOCC6)ncnc5[nH]4)cc3)c2)C1]

In one particular embodiment, the compound is N-[4-[4-(4-morpholinyl)-7H-pyrrolo[2,3-d]pyrimidin-6-yl]phenyl]-4-[[3(S)-[(1-oxo-2-propen-1-yl)amino]-1-piperidinyl]methyl]-2-pyridinecarboxamide, or a pharmaceutically acceptable salt thereof.

In some embodiments, the compound is according to formula (XLIIa).

In some embodiments, the compound is according to formula (XLIIIa).

Embodiments of the compounds of Formula (I) displayed improved potency against menin-MLL with IC₅₀ values of as low as less than 1 nM or less than 0.1 nM, and/or high occupancy of active site of menin (e.g., more than 50%, 70% or 90% occupancy) at low dosages of below 5 mg/kg (e.g., at or below 3 mg/kg) when administered in vivo (e.g., in rats).

In some embodiments, the present invention provides, a pharmaceutical composition comprising a compound according to formula (I).

In some embodiments, the present invention provides, a pharmaceutical composition comprising a therapeutically effective amount of a compound of formula (I), and a pharmaceutically acceptable excipient.

In a particular embodiment, the menin-MLL inhibitor is Compound 10. In a more particular embodiment, the menin-MLL inhibitor is a R-isomer of Compound 10.

In a particular embodiment, the menin-MLL inhibitor is KO-539 or Zifomenib:

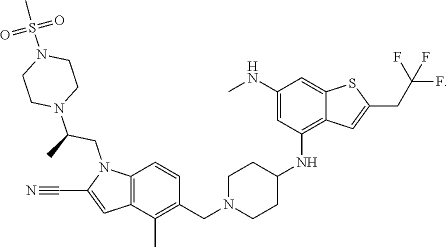 [SMILES: CNc1cc(NC2CCN(Cc3ccc4c(cc(C#N)n4C[C@@H](C)N4CCN(S(C)(=O)=O)CC4)c3C)CC2)c2cc(CC(F)(F)F)sc2c1]

In a particular embodiment, the menin-MILL inhibitor is SNDX-5613 or Revumenib.

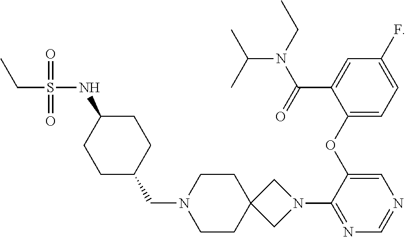 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

Any combination of the groups described above for the various variables is contemplated herein. It is understood that substituents and substitution patterns on the compounds provided herein can be selected by one of ordinary skill in the art to provide compounds that are chemically stable and that can be synthesized by techniques known in the art, as well as those set forth herein.

Further representative embodiments of compounds of Formula (I), include compounds listed in Table 1, or a solvate or a pharmaceutically acceptable salt thereof. The compounds are prepared according to U.S. Pat. No. 11,174,263 B2 or 11,084,825 B2, each of which is incorporated herein by reference in its entirety.

TABLE 1

| ID | Structure | MW |
|---|---|---|
| 1 | 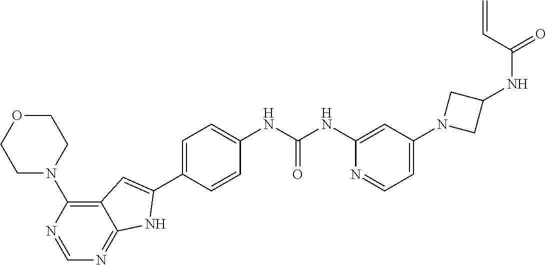 [SMILES: C=CC(=O)NC1CN(c2ccnc(NC(=O)Nc3ccc(-c4cc5c(N6CCOCC6)ncnc5[nH]4)cc3)c2)C1] | 539.59 |
| 2 | 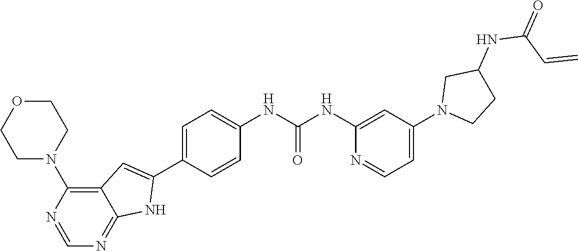 [SMILES: C=CC(=O)NC1CCN(c2ccnc(NC(=O)Nc3ccc(-c4cc5c(N6CCOCC6)ncnc5[nH]4)cc3)c2)C1] | 553.63 |
| 3 | 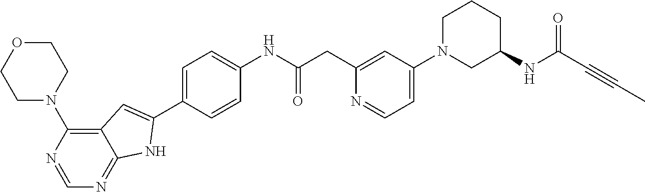 [SMILES: CC#CC(=O)N[C@@H]1CCCN(c2ccnc(CC(=O)Nc3ccc(-c4cc5c(N6CCOCC6)ncnc5[nH]4)cc3)c2)C1] | 578.66 |
| 4 | 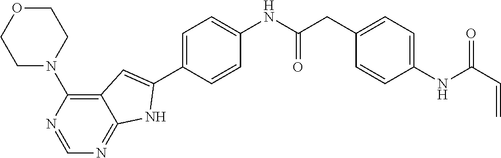 [SMILES: C=CC(=O)Nc1ccc(CC(=O)Nc2ccc(-c3cc4c(N5CCOCC5)ncnc4[nH]3)cc2)cc1] | 482.53 |
| 5 | 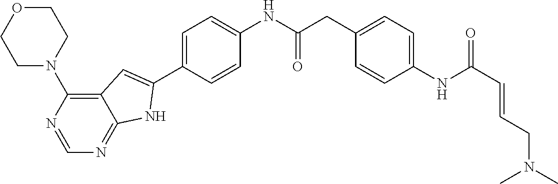 [SMILES: CN(C)C/C=C/C(=O)Nc1ccc(CC(=O)Nc2ccc(-c3cc4c(N5CCOCC5)ncnc4[nH]3)cc2)cc1] | 539.63 |
| 6 | 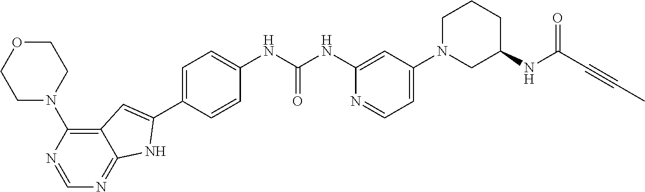 [SMILES: CC#CC(=O)N[C@@H]1CCCN(c2ccnc(NC(=O)Nc3ccc(-c4cc5c(N6CCOCC6)ncnc5[nH]4)cc3)c2)C1] | 579.65 |
| 7 | 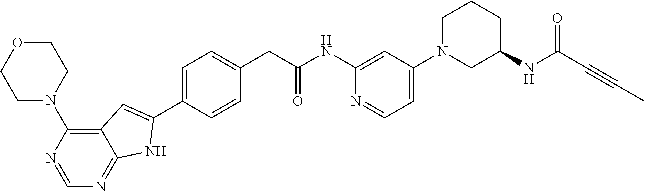 [SMILES: CC#CC(=O)N[C@@H]1CCCN(c2ccnc(NC(=O)Cc3ccc(-c4cc5c(N6CCOCC6)ncnc5[nH]4)cc3)c2)C1] | 578.66 |
| 8 | 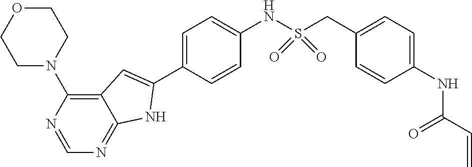 [SMILES: C=CC(=O)Nc1ccc(CS(=O)(=O)Nc2ccc(-c3cc4c(N5CCOCC5)ncnc4[nH]3)cc2)cc1] | 518.59 |
| 9 | 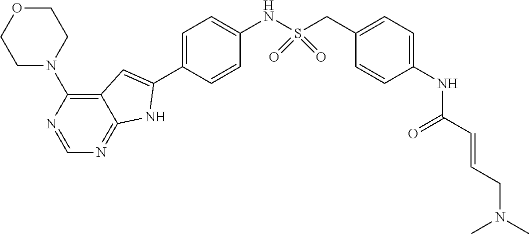 [SMILES: CN(C)C/C=C/C(=O)Nc1ccc(CS(=O)(=O)Nc2ccc(-c3cc4c(N5CCOCC5)ncnc4[nH]3)cc2)cc1] | 575.68 |
| 10 | 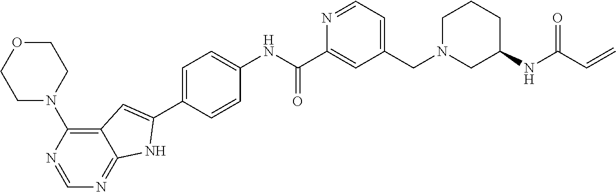 [SMILES: C=CC(=O)N[C@@H]1CCCN(Cc2ccnc(C(=O)Nc3ccc(-c4cc5c(N6CCOCC6)ncnc5[nH]4)cc3)c2)C1] | 566.67 |
| 11 | 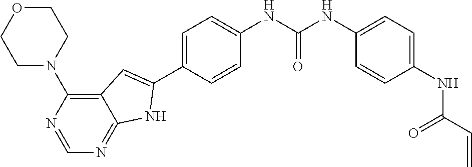 [SMILES: C=CC(=O)Nc1ccc(NC(=O)Nc2ccc(-c3cc4c(N5CCOCC5)ncnc4[nH]3)cc2)cc1] | 483.52 |
| 12 | 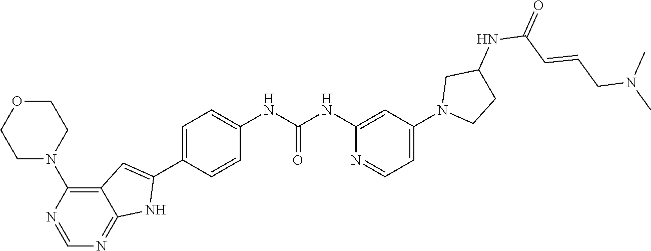 [SMILES: CN(C)C/C=C/C(=O)NC1CCN(c2ccnc(NC(=O)Nc3ccc(-c4cc5c(N6CCOCC6)ncnc5[nH]4)cc3)c2)C1] | 610.72 |
| 13 | 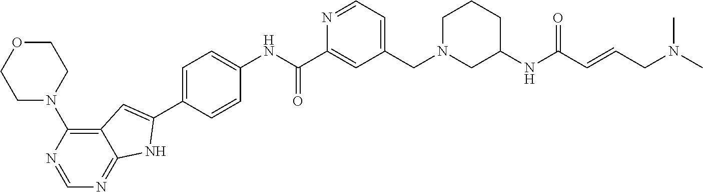 [SMILES: CN(C)C/C=C/C(=O)NC1CCCN(Cc2ccnc(C(=O)Nc3ccc(-c4cc5c(N6CCOCC6)ncnc5[nH]4)cc3)c2)C1] | 623.76 |
| 14 | 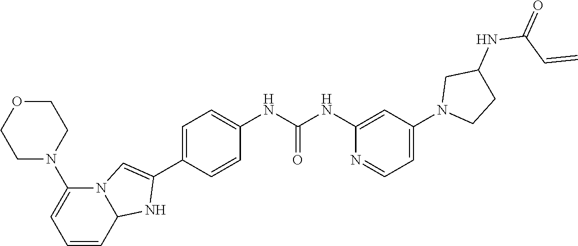 [SMILES: C=CC(=O)NC1CCN(c2ccnc(NC(=O)Nc3ccc(C4=CN5C(N6CCOCC6)=CC=CC5N4)cc3)c2)C1] | 554.65 |
| 15 | 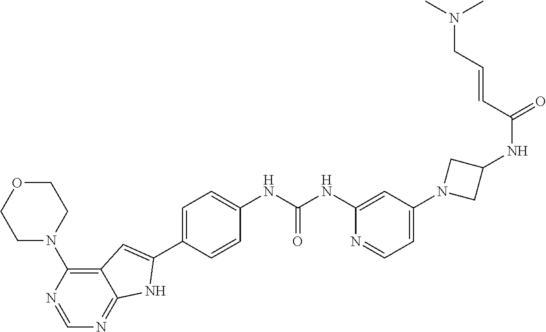 [SMILES: CN(C)C/C=C/C(=O)NC1CN(c2ccnc(NC(=O)Nc3ccc(-c4cc5c(N6CCOCC6)ncnc5[nH]4)cc3)c2)C1] | 596.70 |
| 16 | 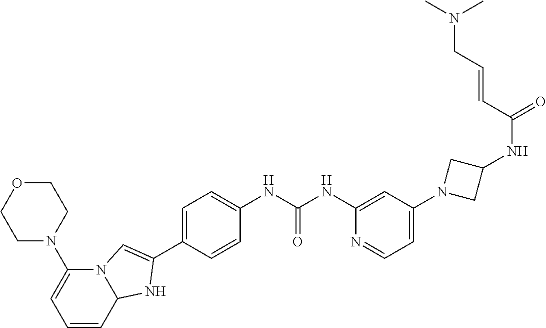 [SMILES: CN(C)C/C=C/C(=O)NC1CN(c2ccnc(NC(=O)Nc3ccc(C4=CN5C(N6CCOCC6)=CC=CC5N4)cc3)c2)C1] | 597.72 |
| 17 | 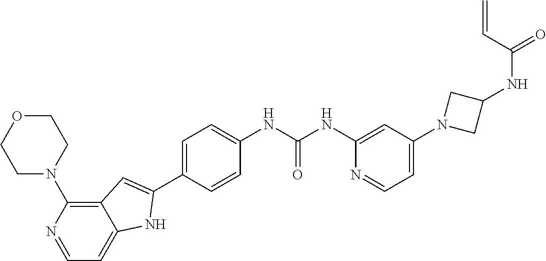 [SMILES: C=CC(=O)NC1CN(c2ccnc(NC(=O)Nc3ccc(-c4cc5c(N6CCOCC6)nccc5[nH]4)cc3)c2)C1] | 538.61 |
| 18 | 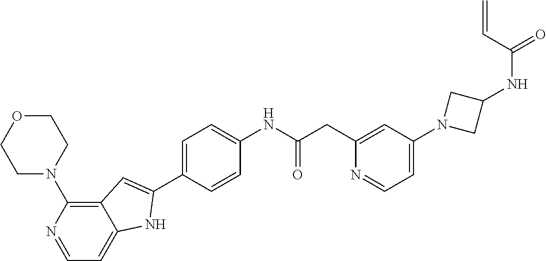 [SMILES: C=CC(=O)NC1CN(c2ccnc(CC(=O)Nc3ccc(-c4cc5c(N6CCOCC6)nccc5[nH]4)cc3)c2)C1] | 537.61 |
| 19 | 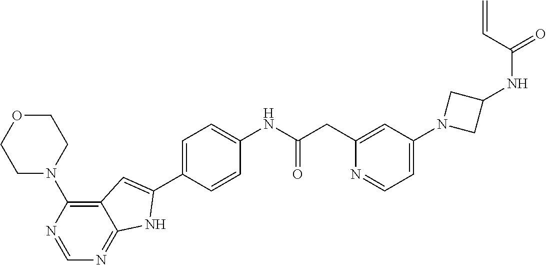 [SMILES: C=CC(=O)NC1CN(c2ccnc(CC(=O)Nc3ccc(-c4cc5c(N6CCOCC6)ncnc5[nH]4)cc3)c2)C1] | 538.60 |
| 20 | 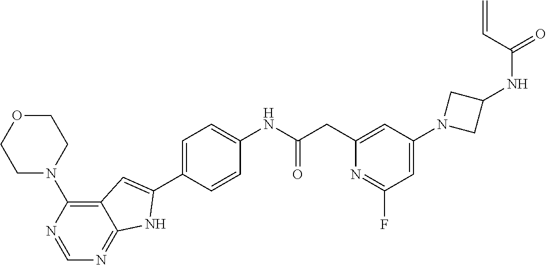 [SMILES: C=CC(=O)NC1CN(c2cc(F)nc(CC(=O)Nc3ccc(-c4cc5c(N6CCOCC6)ncnc5[nH]4)cc3)c2)C1] | 556.59 |
| 21 | 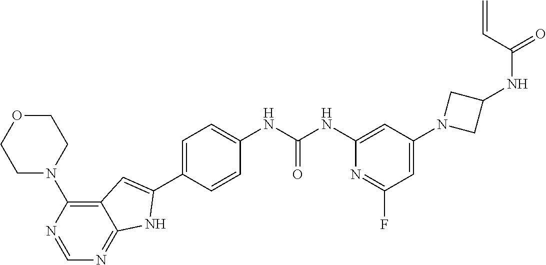 [SMILES: C=CC(=O)NC1CN(c2cc(F)nc(NC(=O)Nc3ccc(-c4cc5c(N6CCOCC6)ncnc5[nH]4)cc3)c2)C1] | 557.58 |
| 22 | 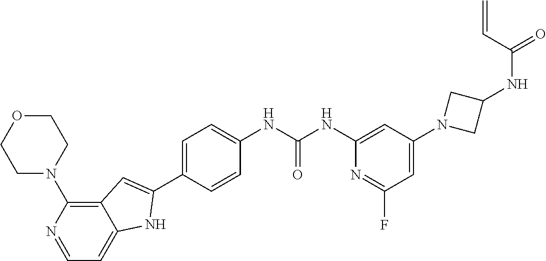 [SMILES: C=CC(=O)NC1CN(c2cc(F)nc(NC(=O)Nc3ccc(-c4cc5c(N6CCOCC6)nccc5[nH]4)cc3)c2)C1] | 556.59 |
| 23 | 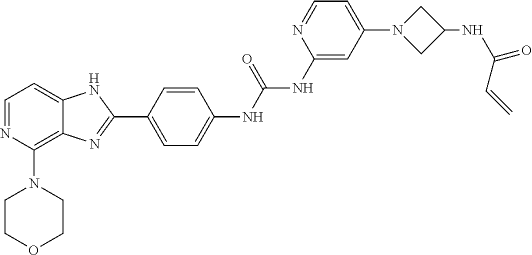 [SMILES: C=CC(=O)NC1CN(c2ccnc(NC(=O)Nc3ccc(-c4nc5c(N6CCOCC6)nccc5[nH]4)cc3)c2)C1] | 539.24 |
| 24 | 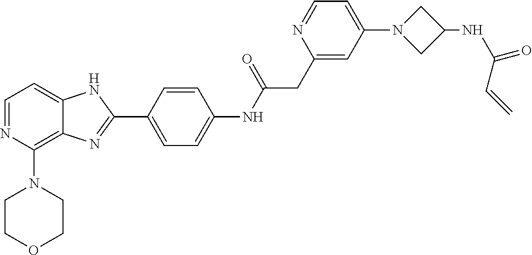 [SMILES: C=CC(=O)NC1CN(c2ccnc(CC(=O)Nc3ccc(-c4nc5c(N6CCOCC6)nccc5[nH]4)cc3)c2)C1] | 538.24 |
| 25 | 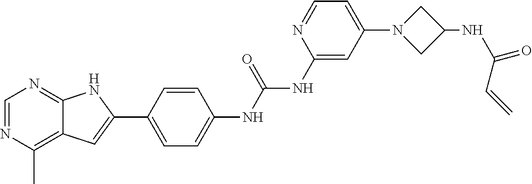 [SMILES: C=CC(=O)NC1CN(c2ccnc(NC(=O)Nc3ccc(-c4cc5c(C)ncnc5[nH]4)cc3)c2)C1] | 468.20 |
| 26 | 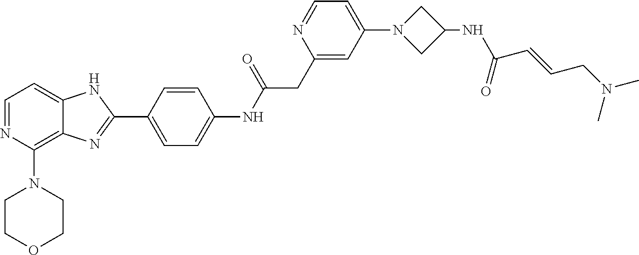 [SMILES: CN(C)C/C=C/C(=O)NC1CN(c2ccnc(CC(=O)Nc3ccc(-c4nc5c(N6CCOCC6)nccc5[nH]4)cc3)c2)C1] | 595.30 |

In some embodiments, provided for use in the compositions and methods described herein is a compound selected from compound Nos. 1-26 in Table 1, or a tautomer thereof, or a pharmaceutically acceptable salt of any of the foregoing. In some embodiments, one or more of the small molecule menin-MLL inhibitors disclosed in U.S. Pat. No. 11,174,263 B2 or 11,084,825 B2, which is incorporated by reference in its entirety, are used in methods described herein.

The disclosure also includes any or all of the stereochemical forms, including any enantiomeric or diastereomeric forms of the compounds described herein, and cis/trans or E/Z isomers. Unless stereochemistry is explicitly indicated in a chemical structure or name, the structure or name is intended to embrace all possible stereoisomers of a compound depicted. In addition, where a specific stereochemical form is depicted, it is understood that all other stereochemical forms are also described and embraced by the disclosure, as well as the general non-stereospecific form and mixtures of the disclosed compounds in any ratio, including mixtures of two or more stereochemical forms of a disclosed in any ratio, such that racemic, non-racemic, enantioenriched and scalemic mixtures of a compound are embraced. Compositions comprising a disclosed compound also are intended, such as a composition of substantially pure compound, including a specific stereochemical form thereof. Compositions comprising a mixture of disclosed compounds in any ratio also are embraced by the disclosure, including compositions comprising mixtures of two or more stereochemical forms of a disclosed compound in any ratio, such that racemic, non-racemic, enantioenriched, and scalemic mixtures of a compound are embraced by the disclosure. If stereochemistry is explicitly indicated for one portion or portions of a molecule, but not for another portion or portions of a molecule, the structure is intended to embrace all possible stereoisomers for the portion or portions where stereochemistry is not explicitly indicated. The disclosure embraces any and all tautomeric forms of the compounds described herein.

The disclosure embraces all salts of the compounds described herein, as well as methods of using such salts of the compounds. In one or more embodiments, the salts of the compounds comprise pharmaceutically acceptable salts. Pharmaceutically acceptable salts are those salts that can be administered as drugs or pharmaceuticals to humans and/or animals and that, upon administration, retain at least some of the biological activity of the free compound (neutral compound or non-salt compound). The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, also can be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N′-dibenzylethylenediamine, and triethylamine salts. Salts of acidic compounds with amino acids, such as lysine salts, also can be prepared. For lists of pharmaceutically acceptable salts, see, for example, P. H. Stahl and C. G. Wermuth (eds.) “Handbook of Pharmaceutical Salts, Properties, Selection and Use” Wiley-VCH, 2011 (ISBN: 978-3-90639-051-2). Several pharmaceutically acceptable salts are also disclosed in Berge, J. Pharm. Sci. 66: 1 (1977).

In certain embodiments, the menin-MLL inhibitors of the disclosure are administered orally, intravenously, subcutaneously, or by pulmonary administration in vivo, in an individual undergoing autologous cell-based therapy, or administered orally, intravenously, subcutaneously or by pulmonary administration to a donor providing pancreatic beta cells for allogeneic cell-based therapy.

### Harvesting Cells

In the cell therapy methods described herein, cells are harvested from a donor. The cells can be any pancreatic beta cells deemed suitable by the person of skill. The harvesting can be according to standard techniques. In certain embodiments, pancreatic beta cells are harvested from a donor.

In certain embodiments, one or more menin-MLL inhibitors enhance the harvesting step. In certain embodiments, one or more menin-MLL inhibitors activate pancreatic beta cells in vivo prior to harvesting. In such embodiments, one or more menin-MLL inhibitors are administered to a donor in an amount sufficient to enhance the desired cells for harvest. In certain embodiments, one or more menin-MLL inhibitors are administered to an individual prior to harvest. In certain embodiments, the menin-MLL inhibitor enhances in vivo activation. In certain embodiments, the menin-MLL inhibitor enhances in vivo differentiation. In certain embodiments, the menin-MLL inhibitor enhances in vivo stimulation. In certain embodiments, the menin-MLL inhibitor enhances in vivo priming of cells.

The dose of the menin-MLL inhibitor can be any dose deemed suitable by the person of skill. In certain embodiments, the dose is effective to enhance the cells desired for harvest. In certain embodiments, the dose is between In certain embodiments, the dose is selected from 25 mg to 1000 mg, 25 mg to 750 mg, 25 mg to 650 mg, and 25 mg to 500 mg. In certain embodiments, the dose of compound A is selected from 25 mg, 50 mg, 75 mg, 100 mg, 125 mg, 150 mg, 175 mg, 200 mg, 325 mg, 500 mg, and 650 mg. In certain embodiments, the dose is administered daily. In certain embodiments, the dose is administered twice per day. In certain embodiments, the dose is administered three times per day. In certain embodiments, the dose is administered four times per day. In certain embodiments, the dose is administered daily in divided doses.

The dose is administered for a period of time on a schedule deemed suitable by the person of skill. In some embodiments, the dose is administered for about 1 day, about 2 days, about 3 days, about 4 days, about 5 days, about 6 days or about 7 days, 1 to 2 days, 1 to 3 days, 1 to 4 days, 1 to 5 days, 1 to 6 days, 1 to 7 days, 1 to 10 days, 1 to 14 days, 1 to 21 days, 1 to 28 days, 1 to 45 days, or 1 to 60 days. In certain embodiments, the dose is administered daily for a cycle of 28 days.

In certain embodiments, the harvested cells are formulated in cryopreservation media and placed in cryogenic storage units such as liquid nitrogen freezers (−195° C.) or ultra-low temperature freezers (−65° C., −80° C., or −120° C.) for long term storage of at least one month, 2 months, 3 months, 4 months, 6 months, 1 year, 2 years, 3 years, or at least 5 years. In some embodiments, thawed cells are expanded by methods described herein.

In certain embodiments, the harvested cell population is isolated or cultured under selective conditions wherein certain types of pancreatic beta cells are enriched prior to the cell population being expanded ex vivo or in vitro.

### Proliferation of Pancreatic Beta Cells

In certain embodiments, the pancreatic beta cells described herein are proliferated in culture by any method deemed suitable by the person of skill. Standard techniques are useful here.

In certain methods, cells undergoing proliferation are cultured in the presence of one or more agents or compounds to facilitate propagation, including enrichment of cells of particularly desired maturation levels prior to being infused into an individual in need thereof. Useful compounds include growth factors, such as TGF-β. Other useful compounds include agents that can modify a surface to minimize immune reactions and tissue rejection in the recipient.

In certain embodiments, the methods comprise proliferation in the presence of a menin-MLL inhibitor. In certain embodiments, pancreatic beta cells are cultured ex vivo or in vitro in the presence of a menin-MLL inhibitor, at a concentration of between 1 pM to about 100 mM, about 0.001 pM to about 100 μM, about 0.01 pM to about 50 μM, about 0.001 nM to about 50 μM, about 100 nM to about 10 μM, about 0.01 μM to about 25 μM, about 0.1 μM to about 15 μM, about 1 μM to about 10 μM, about 0.1 pM to about 100 nM, about 1 pM to about 10 nM, or about 1 pM to about 1 nM.

In certain methods, the cells undergoing proliferation in the presence of menin-MLL inhibitor are provided additional menin-MLL inhibitor to replenish the amount used by the cells during culture. In some embodiments, the menin-MLL inhibitor is replenished during single proliferation. In other embodiments, the menin-MLL inhibitor is replenished during double proliferation but only during the selective proliferation phase. In other embodiments, the menin-MLL inhibitor is replenished during proliferation but only during the second proliferation phase. In other embodiments, the menin-MLL inhibitor is replenished during proliferation during both the selective proliferation phase and the second proliferation phase. In other embodiments, the menin-MLL inhibitor is added to the culture every 2, 3, 4, 5, 6, 7, 8, 9, 10, or 11 days during proliferation (whether during a single proliferation, selective proliferation, second proliferation or both selective proliferation and second proliferation). In other embodiments, the menin-MLL inhibitor is replenished only once, twice, or three times during proliferation (whether during a single proliferation, selective proliferation, second proliferation, or both selective proliferation and second proliferation).

In certain embodiments, pancreatic beta cells are subjected to proliferation using methods described herein for a period of from about 1 day to about 48 days, about 1 day to about 28 days, about 1 days to about 24 days, or about 1 day to about 14 days.

In one or more embodiments, a composition includes beta cells (or expanded pancreatic beta cells) with improved beta cell function. For example, beta cell function may be improved by an increase in beta cell insulin content, glucose-stimulated insulin secretion, or both, compared to a control. In one or more embodiments, a method includes inducing improved beta cell function. For example, beta cell function may be improved in one or more embodiments of a method herein, compared to when one or more embodiments of a method herein is not used. Such methods may result in an increase in beta cell insulin content, glucose-stimulated insulin secretion, or both, compared to when a method of one or more embodiments is not used.

### Methods of Treatment

In the cell therapy methods provided herein, expanded cells are administered to a patient in need thereof. In certain embodiments, the patient is the same as the donor. In certain embodiments, the patient and the donor are not the same individual. In certain embodiments, the donor is xenogeneic, and the patient is human. In some embodiments, patients are administered autologous cells according to methods described herein. In some embodiments, patients are administered allogeneic cells according to methods described herein.

In certain embodiments, the methods comprise administering to an individual in need of treatment, a composition comprising an effective amount of the pancreatic beta cells that have been produced ex vivo or in vitro as provided herein. Therapeutically effective doses of the infusion population can be in the range of about one million to about 200 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges. In some embodiments, the method comprises administering between 2×10⁶ and 2×10⁸ viable pancreatic beta cells per kg of body weight.

The infusion population and compositions thereof can be administered to an individual in need thereof using standard administration techniques, formulations, and/or devices. Provided are formulations and administration with devices, such as syringes and vials, for storage and administration of the compositions. Formulations or pharmaceutical composition comprising the pancreatic beta cells include those for intravenous, intraperitoneal, subcutaneous, intramuscular, or pulmonary administration. In some embodiments, the pancreatic beta cells are administered parenterally. The term “parenteral,” as used herein, includes intravenous, intramuscular, subcutaneous, rectal, vaginal, and intraperitoneal administration. In some embodiments, the cell populations are administered to a subject using peripheral systemic delivery by intravenous, intraperitoneal, or subcutaneous injections. Compositions of the modified pancreatic beta cells can be provided as sterile liquid preparations, e.g., isotonic aqueous solutions, suspensions, emulsions, dispersions, or viscous compositions, which may in some aspects be buffered to a selected pH. Viscous compositions can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the pancreatic beta cells in a solvent, such as in admixture with a suitable carrier, diluent, or excipient such as sterile water, physiological saline, glucose, dextrose, or the like.

In certain embodiments, the cell therapy is administered in combination with one or more menin-MLL inhibitors. For instance, in some therapeutic regimens of the present disclosure, both the modified pancreatic beta cells and a menin-MLL inhibitor are administered to a subject in need thereof, wherein the menin-MLL inhibitor is a compound described herein or a pharmaceutically acceptable salt or solvate thereof.

In some embodiments, one or more of the small molecule menin-MLL inhibitors disclosed in U.S. Pat. No. 11,174,263 B2 or 11,084,825 B2, each of which is incorporated by reference in its entirety, are used in a treatment method described herein. In certain embodiments, the menin-MLL inhibitor is according to Formula (I). In certain embodiments, the menin-MLL inhibitor is selected from compounds 1-26.

In certain embodiments, menin-MLL inhibitors are administered to a patient receiving therapeutic cells. The dose of the menin-MLL inhibitor can be any dose deemed suitable by the person of skill. In certain embodiments, the dose is effective to enhance the cells desired for harvest. In certain embodiments, the dose is between In certain embodiments, the dose is selected from 25 mg to 1000 mg, 25 mg to 750 mg, 25 mg to 650 mg, and 25 mg to 500 mg. In certain embodiments, the dose of compound A is selected from 25 mg, 50 mg, 75 mg, 100 mg, 125 mg, 150 mg, 175 mg, 200 mg, 325 mg, 500 mg, and 650 mg. In certain embodiments, the dose is administered daily. In certain embodiments, the dose is administered twice per day. In certain embodiments, the dose is administered three times per day. In certain embodiments, the dose is administered four times per day. In certain embodiments, the dose is administered daily in divided doses.

The dose is administered for a period of time on a schedule deemed suitable by the person of skill. In some embodiments, the dose is administered for about 1 day, about 2 days, about 3 days, about 4 days, about 5 days, about 6 days or about 7 days, 1 to 2 days, 1 to 3 days, 1 to 4 days, 1 to 5 days, 1 to 6 days, 1 to 7 days, 1 to 10 days, 1 to 14 days, 1 to 21 days, 1 to 28 days, 1 to 45 days, or 1 to 60 days. In certain embodiments, the dose is administered daily for a cycle of 28 days.

Post Infusion Treatment with a Menin-MLL Inhibitor

In certain embodiments, the menin-MLL inhibitor is administered to an individual after an infusion of pancreatic beta cells. The length of time between infusion of the pancreatic beta cells and the administration of the menin-MLL inhibitor, or vice versa, can be from about 1 minute to about 1 hour, about 5 minutes to about 1 hour, about 10 minutes to about 1 hour, about 15 minutes to about 1 hour, about 20 minutes to about 1 hour, about 30 minutes to about 1 hour, about 45 minutes to about 1 hour, about 1 hour to about 2 hours, about 1 hour to about 4 hours, about 1 hour to about 6 hours, about 1 hour to about 8 hours, about 1 hour to about 12 hours, about 1 hour to about 24 hours, about 2 hours to about 24 hours, about 6 hours to about 7 hours, about 6 hours to about 24 hours, about 8 hours to about 24 hours, about 10 hours to about 24 hours, about 15 hours to about 24 hours, about 20 hours to about 24 hours, about 12 hours to about 48 hours, about 24 hours to about 48 hours, or about 36 hours to about 48 hours.

In some embodiments, the menin-MLL inhibitor is administered as supportive therapy post infusion. In some embodiments, the dose of the menin-MLL inhibitor can be any dose deemed suitable by the person of skill. In certain embodiments, the dose is effective to enhance the cells desired for harvest. In certain embodiments, the dose is between In certain embodiments, the dose is selected from 25 mg to 1000 mg, 25 mg to 750 mg, 25 mg to 650 mg, and 25 mg to 500 mg. In certain embodiments, the dose of compound A is selected from 25 mg, 50 mg, 75 mg, 100 mg, 125 mg, 150 mg, 175 mg, 200 mg, 325 mg, 500 mg, and 650 mg. In certain embodiments, the dose is administered daily. In certain embodiments, the dose is administered twice per day. In certain embodiments, the dose is administered three times per day. In certain embodiments, the dose is administered four times per day. In certain embodiments, the dose is administered daily in divided doses.

The dose is administered for a period of time on a schedule deemed suitable by the person of skill. In some embodiments, the dose is administered for about 1 day, about 2 days, about 3 days, about 4 days, about 5 days, about 6 days or about 7 days, 1 to 2 days, 1 to 3 days, 1 to 4 days, 1 to 5 days, 1 to 6 days, 1 to 7 days, 1 to 10 days, 1 to 14 days, 1 to 21 days, 1 to 28 days, 1 to 45 days, or 1 to 60 days. In certain embodiments, the dose is administered daily for a cycle of 28 days.

### Conditions Treated

The methods and compositions herein can be used for the treatment of any condition deemed suitable by the person of skill. In certain embodiments, the condition is a pancreatic disease or disorder. In certain embodiments, the condition is diabetes. In some embodiments, using a compound as disclosed herein, the disease or condition to be treated is diabetes mellitus. In some embodiments, the disease or condition is type 1 diabetes mellitus. In some embodiments, the disease or condition is type 2 diabetes mellitus. In some embodiments, the disease or condition is gestational diabetes mellitus. In some embodiments, the disease or condition is maturity onset diabetes of the young. In some embodiments, the disease or condition is steroid diabetes. In some embodiments, the disease or condition is double diabetes. In some embodiments, the disease or condition is a metabolic condition. In some embodiments, the disease or condition is a genetic disorder. In some embodiments, the disease or condition is a metabolic condition affected by beta cells. In some embodiments, the disease or condition is a genetic disorder affected by beta cells.

### Pharmaceutical Compositions and Dosage Forms

In some embodiments, the menin-MLL inhibitors of the disclosure are formulated as pills, capsules, tablets, syrups, ampules, lozenges, powders for oral administration to an individual. In some embodiments, the menin-MLL inhibitors are formulated for infusion or injection. In some embodiments, the menin-MLL inhibitors are formulated for use in cell culture in vitro or ex vivo. In some embodiments of the disclosure the menin-MLL inhibitor provided herein is a pharmaceutical composition or single unit dosage form. Pharmaceutical compositions and single unit dosage forms provided herein comprise a prophylactically or therapeutically effective amount of one or more menin-MLL inhibitors. In some embodiments, one or more of the small molecule menin-MLL inhibitors disclosed in U.S. Pat. No. 11,174,263 B2 or 11,084,825 B2, each of which is incorporated by reference in its entirety.

Cells produced by the methods provided herein can be formulated and administered according to standard techniques. Briefly, pharmaceutical compositions may comprise a cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose, dextrans, or mannitol; proteins; polypeptides or amino acids such as glycine; antioxidants; chelating agents such as EDTA or glutathione; adjuvants (e.g., aluminum hydroxide); and preservatives. In certain embodiments, cell compositions are formulated for intravenous administration. In certain embodiments, cell compositions are formulated for encapsulation. Pharmaceutical compositions of the present invention may be administered in a manner appropriate to the disease to be treated (or prevented). The quantity and frequency of administration will be determined by such factors as the condition of the patient, and the type and severity of the patient's disease, and appropriate dosages may be determined by clinical trials.

### EXAMPLES

### Example 1: Effect of Compounds on Human Beta Cell Proliferation and Function

### Beta Cell Proliferation Using Human Islet Microtissue:

The ability of menin-MLL inhibitors to induce beta cell proliferation is tested on human islet microtissue preparations (InSphero). Briefly, human islet microtissues are produced by enzymatic dissociation of the primary islet cells followed by controlled scaffold-free hanging-drop-based self-reaggregation of the islet cells (Misun et al., 2020, Adv. Biosyst. 2020, 4, 1900291). The process helps eliminate contaminating exocrine cells, while enabling homogenous and native-like distribution of endocrine cells within each islet microtissue. Islet microtissues generated by this process are uniform in size, and cellular composition, long lived, functionally robust and display long-term and stable functionality and viability during in vitro culture (Misun et al., 2020, Adv. Biosyst. 2020, 4, 1900291). The methodology confirms primary human beta cells can be cultured long term (few weeks) under ex-vivo culture conditions to enable beta cell expansion ex-vivo.

### Microtissue Culture and Compound Dosing:

Islet microtissues (MTs) were aggregated for 5 days and then released into ultra-low attachment plates. Half of the plates were maintained in standard culture medium (5.5 mM glucose), and half were cultured in high-glucose culture medium (GTX, 8 mM glucose) starting at Day 9, for the duration of the whole experiment. Starting on Day 12 (after 3 days of GTX pre-treatment for the relevant plates), dosing with compound 10, a representative irreversible menin-MLL inhibitor, was initiated, using a Tecan D300e Digital Dispenser for harmine and Biomea compound. DMSO was normalized by volume across all wells. Media was exchanged and compounds redosed every 2-3 days throughout the experiment. EdU was included during the final 4 days of each treatment period. Compound dosing was randomized; to avoid compound cross-contamination between wells, all media exchanges were performed using 96-well deep-well plates with pipette tip exchange.

### Functional Assay:

At the described termination point (1, 2, or 3 weeks), culture media supernatants were collected for measurement of chronic insulin secretion (insulin secreted since last medium exchange, i.e. 72 hours in this project). MTs were washed once with Krebs Ringer Hepes Buffer (KRHB) containing 2.8 mM glucose, then equilibrated in KRHB containing 2.8 mM glucose for 1 hour. Glucose stimulated insulin secretion assays were then performed by incubating MTs with KRBH containing 2.8 mM glucose for 2 hours, followed by supernatant collection and incubation with KRHB containing 16.7 mM glucose for 2 hours and supernatant collection. Tissues were next analyzed for total ATP content using the Promega CellTiter-Glo® Luminescent Cell Viability Assay. Lysates were also used for measurement of total insulin content. Supernatants and lysates were appropriately diluted and total and secreted insulin were quantified using ALPCO Stellux® Chemi Human Insulin ELISA.

### 3D Staining and Microscopy Analysis:

Proliferating cells were labeled in 3D by incubating MTs with 10 M EdU during the final 4 days of compound treatment. At the described termination point (1, 2, or 3 weeks), MTs were washed twice with PBS, fixed for 15 min in 4% PFA, washed twice more with PBS and stored in PBS with 0.05% sodium azide until staining. Islet MTs were then permeabilized with permeabilization buffer (Triton® X-100, 0.5% in PBS w/o Mg2+Ca2+) and washed twice with PBS. EdU was labeled using the Click-it reaction (Click-iT™ EdU Alexa Fluor™ 647 HCS Assay, Thermo Fisher). MTs were washed twice with PBS and blocked with 10% FCS solution to prevent nonspecific antibody binding, before overnight incubation with rabbit mAB NKX6.1 [EPR20405] (Abcam, ab221549) at a 1:200 dilution in antibody dilution buffer (10% FCS, 0.2% Triton® X-100 in PBS w/o Mg2+Ca2+) as a 3-cell marker. Goat anti-rabbit secondary antibody AF568 (ThermoFisher A11036) was used as secondary antibody at a 1:200 dilution in antibody dilution buffer, along with DAPI. Finally, islet MTs were transferred into Akura 384-well plates and cleared with ScaleS4 for 3D imaging. Images were acquired using the Yokogawa CQI Benchtop High-Content Analysis System, taking fluorescent images in 3 m z-steps in blue, red and far-red channels. DAPI-, NKX6.1- and EdU-positive nuclei and signal colocalization were quantified in 3D using a customized CellPathfinder pipeline. Proliferating cells and proliferating-cells were manually counted to validate automated results. Statistical significance was determined with One-way ANOVA and Dunnett's multiple comparisons test, rejecting the null hypothesis at p=0.05. Outliers were detected with ROUT's outlier test (Q=5%). No outlier test was applied to the proliferating cells.

### Induction of Human Beta Cell Proliferation by Compounds

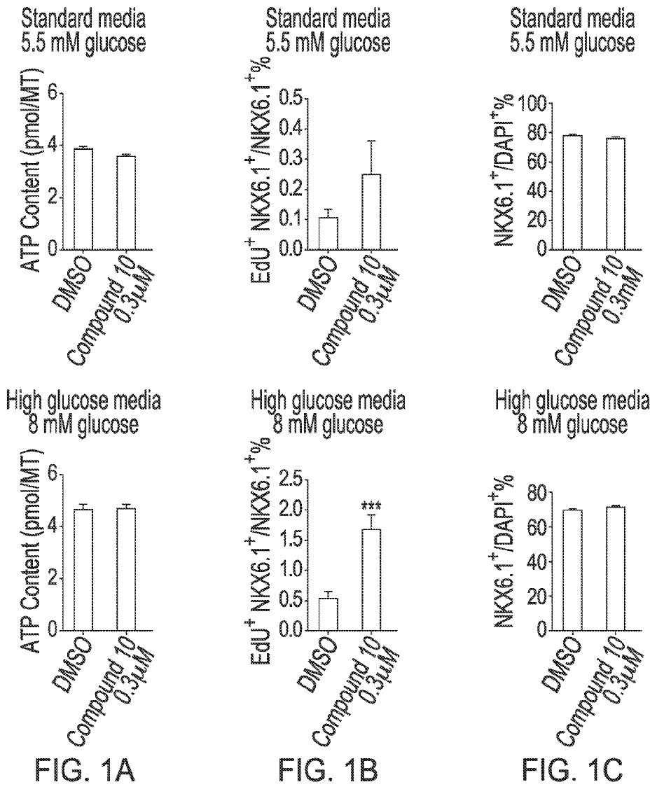

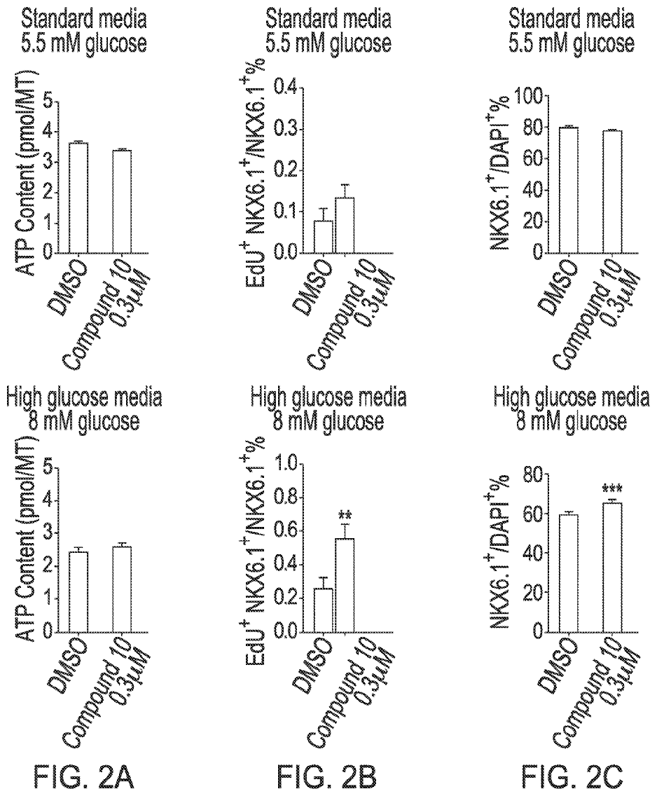

FIGS. 1 and 2 provide human pancreatic islet beta cell proliferation after 14 days (FIG. 1) and 21 days (FIG. 2) of culturing in the presence of compound 10 (0.3 μM). In FIGS. 1 and 2, A is ATP content; B is proliferating beta cell fraction (EdU⁺NKX6.1⁺/NKX6.1%); and C is beta cell fraction (NKX6.1⁺/DAPI⁺ %). Upper graphs are standard media, and lower graphs are high glucose media. Data represents mean±SEM of 1 donor with n=4-10 technical replicates. One-way ANOVA with Dunnett's posthoc test relative to DMSO control. * p<0.05; ** p<0.01; and *** p<0.001. Compound 10 induced human pancreatic islet beta cells substantially compared to control (graph(s) B) on Day 14 and on Day 21.

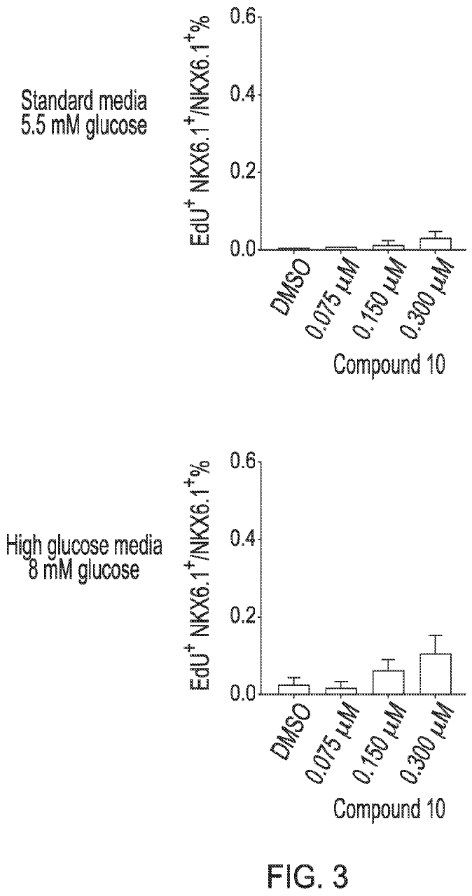

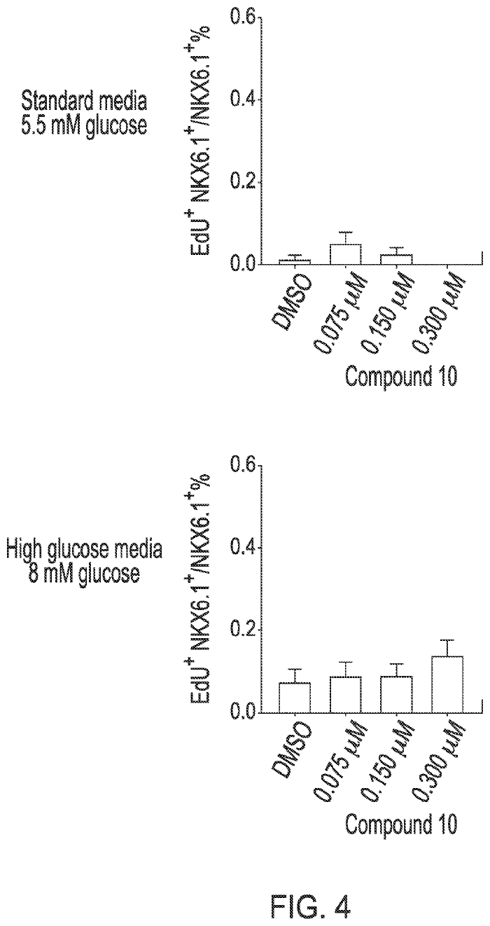

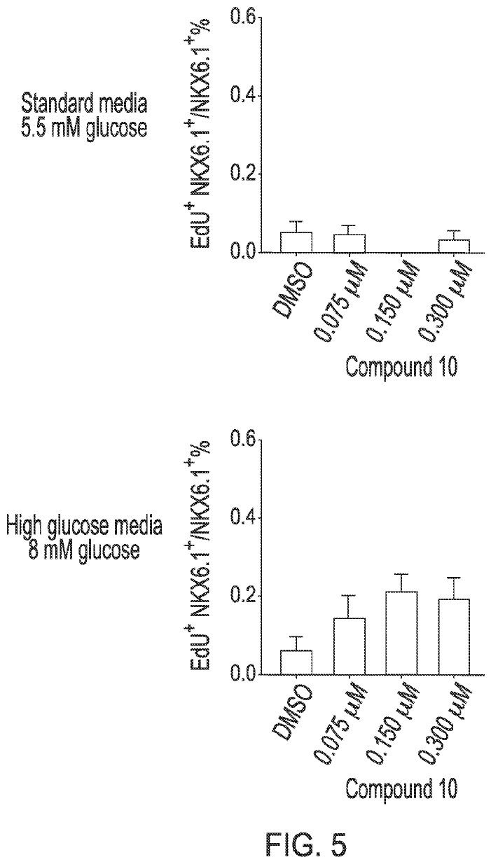

FIGS. 3, 4, and 5 provide human pancreatic islet beta cell proliferation after 7 days (FIG. 3), 14 days (FIG. 4), and 21 days (FIG. 5) of culturing in the presence of compound 10 (0.075 μM, 0.0150 μM, and 0.3 μM). Upper graphs are standard media (5 mM glucose), and lower graphs are high glucose media (8 mM glucose). Data represents mean±SEM of 1 donor with n=4-10 technical replicates. One-way ANOVA with Dunnett's posthoc test relative to DMSO control. * p<0.05; ** p<0.01; and *** p<0.001.

The present example demonstrates that Compound 10 induces improved beta cell function as well as substantial proliferation of human pancreatic islet beta cells. In high glucose medium, compound 10 treatment induced a dose dependent increase in β-cell proliferation relative to solvent control that was more pronounced than in standard medium. An increase in proliferation was noted at all three concentrations and reached highest by day 21. Total cell proliferation was not increased with compound 10 treatment compared to DMSO control, suggesting high μ-cell specificity. Total β-cell count and β-cell fraction were significantly increased in a dose-dependent manner. Islet microtissues treated with compound 10 showed good viability through the duration of the assay, as revealed by ATP content readout.

In addition to promoting selective proliferation of human islet beta cells, compound 10 also improved beta cell function as observed by compound-induced increase in beta cell insulin content and glucose-stimulated insulin secretion. Under high glucose conditions, human islet microtissues treated for 2 and 3 weeks with compound 10 showed a dose dependent increase in beta cell insulin content, an effect not observed with islets treated with vehicle control (DMSO). Compound 10 induced increase in insulin was not observed under standard glucose culture conditions, where total insulin content remains largely unchanged.

Under high glucose conditions Compound 10 improved glucose stimulated insulin secretion, compared to vehicle treatment. This effect is not observed under standard glucose culture conditions.

### Example 2: Proliferation of Human Beta Cell Line (EndoC-βH5) in Presence of Compound 10

The recently developed human EndoC-βH5 cell line closely recapitulates properties of human β-cells in vivo, including improved insulin secretion and response to GLP1R agonists, in the absence of proliferation (Human Cell Design).

EndoC-βH5 cells obtained as cryopreserved cells from manufacturer (Human Cell Design) are thawed and cultured following the manufacturer's protocol. Cells were seeded 20,000 cells/well into 384-well plates (Szczerbinska et al., 2022, Biomedicines 2022 10(1):103) or at higher densities in 12-well or 96-well culture plates. Cells are cultured for up to 4-6 weeks in the presence of Compound 10 at the appropriate concentration. Compound containing media is replenished twice a week.

All publications and patent, applications cited in this specification are herein incorporated by reference as if each individual publication or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof. Accordingly, it is intended that the scope of the subject matter limited solely by the scope of the following claims, including equivalents thereof.

1. A method of producing expanded pancreatic beta cells, comprising the step of:
proliferating pancreatic beta cells in vitro in the presence of a menin-MLL inhibitor in culture under conditions wherein a substantial number of expanded pancreatic beta cells are produced.

2. A method of producing expanded pancreatic beta cells, comprising the step of:
contacting a population of pancreatic beta cells with a sufficient amount of a menin-MLL inhibitor to increase the number of cells in the population.

3. The method of claim 2, wherein the contacting is in vivo.

4. The method of claim 2, wherein the contacting is in vivo by administering the menin-MLL inhibitor to a subject capable of producing pancreatic beta cells.

5. The method of claim 2, wherein the contacting is ex vivo or in vitro.

6. The method of claim 1, wherein the pancreatic beta cells are harvested from a donor.

7. The method of claim 1, wherein the culture comprises TGF-β.

8. The method of claim 1 that produces at least 10⁶, 10⁷, 10⁸, 10⁹, 10¹⁰, or 10¹¹ expanded pancreatic beta cells.

9. The method of claim 1, wherein the expanded pancreatic beta cells are capable of use in therapy without further proliferation.

10. The method of claim 1, further comprising harvesting the expanded pancreatic beta cells.

11. The method of claim 10, further comprising administering the harvested pancreatic beta cells to a patient in need thereof.

12. The method of claim 11, for the treatment of a pancreatic condition.

13. The method of claim 11, for the treatment of diabetes mellitus.

14. The method of claim 11, further comprising administering an effective amount of a menin-MLL inhibitor to the patient to enhance the therapeutic effect of the pancreatic beta cells.

15. The method of claim 1, wherein the cells are human pancreatic beta cells.

16. The method of claim 1, wherein the cells are human neonatal pancreatic beta cells.

17. The method of claim 1, wherein the cells are stem cell derived beta cells.

18. The method of claim 1, wherein the menin-MLL inhibitor is
i) a compound according to formula (I): 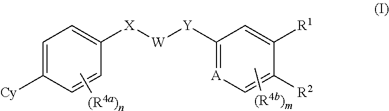
or a pharmaceutically acceptable salt thereof,
wherein:
A is C or N;
Cy is substituted or unsubstituted 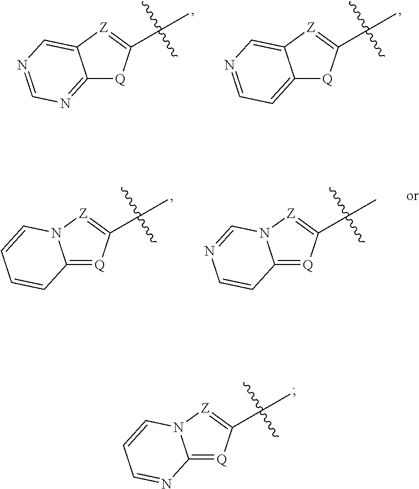
Q is N, —N(H)—, —O—, or —S—;
Z is —CR^{5a}═ or —N═;
X is —NR^{3a}—, —C(R^{3b})₂—, or —O—;
Y is a single bond, —NR^{3a}—, —C(R^{3b})₂—, or —O—;
W is —C(O)—, —S(O)—, or —S(O)₂—;
one of R¹ and R² is Cy²-N(H)C(O)—C(R^{6a})═C(R^{6b})(R^{6c}), or CH₂-Cy²-N(H)C(O)—C(R^{6a})═C(R^{6b})(R^{6c}); and the other is H, C₁₋₆ alkyl, C₁₋₆ haloalkyl, halo, or CN;
Cy² is an unsubstituted group or a substituted group selected from phenyl, pyridyl, or a 4-7 membered heterocycloalkyl ring having 1-2 heteroatoms independently selected from nitrogen, oxygen, or sulfur;
each R^{3a}, and R^{3b} is independently H or C₁₋₆ alkyl;
each R^{4a} and R^{4b} is independently H, halo, CN, OR, —N(R)₂, —C(O)N(R)₂, —NRC(O)R, —SO₂R, —C(O)R, —CO₂R, or an unsubstituted group or a substituted group selected from C₁₋₆ alkyl, C₃₋₇ cycloalkyl, a 4-7 membered heterocycloalkyl ring having 1-2 heteroatoms independently selected from nitrogen, oxygen, or sulfur, phenyl, an 8-10 membered bicyclic aryl ring, and a 5-6 membered heteroaryl ring having 1-4 heteroatoms independently selected from nitrogen, oxygen, or sulfur;
each R is independently H, or an unsubstituted group or a substituted group selected from C₁₋₆ aliphatic, phenyl, an 8-10 membered bicyclic aryl ring, a 4-7 membered saturated or partially unsaturated heterocyclic ring having 1-2 heteroatoms independently selected from nitrogen, oxygen, or sulfur, and a 5-6 membered heteroaryl ring having 1-4 heteroatoms independently selected from nitrogen, oxygen, or sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, or sulfur;
R^{5a} is H, C₁₋₆ alkyl, C₁₋₆ haloalkyl, halo, or CN;
each R^{6a} and R^{6b} is independently H or C₁₋₆ alkyl; or R^{6a} and R^{6b} are joined together to form a bond;
R^{6c} is H or substituted or unsubstituted C₁₋₆ alkyl;
m is 1, 2, or 3; and
n is 1, 2, 3, or 4;
or
ii) the compound is KO-539 or Zifomenib 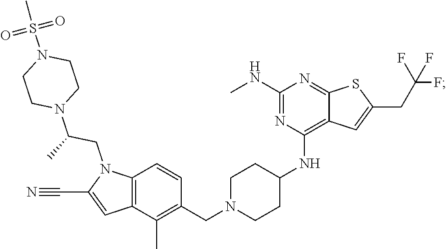 [SMILES: CNc1nc(NC2CCN(Cc3ccc4c(cc(C#N)n4C[C@H](C)N4CCN(S(C)(=O)=O)CC4)c3C)CC2)c2cc(CC(F)(F)F)sc2n1]
or
iii) the compound is SNDX-5613 or Revumenib

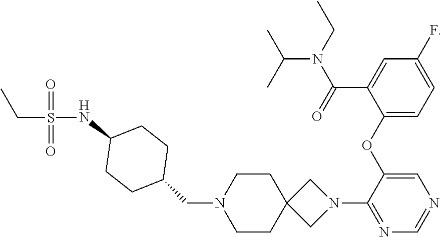 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

19. The method of claim 18, wherein the menin-MLL inhibitor is a compound selected from the group consisting of compounds 1-26, and pharmaceutically acceptable salts and solvates thereof:

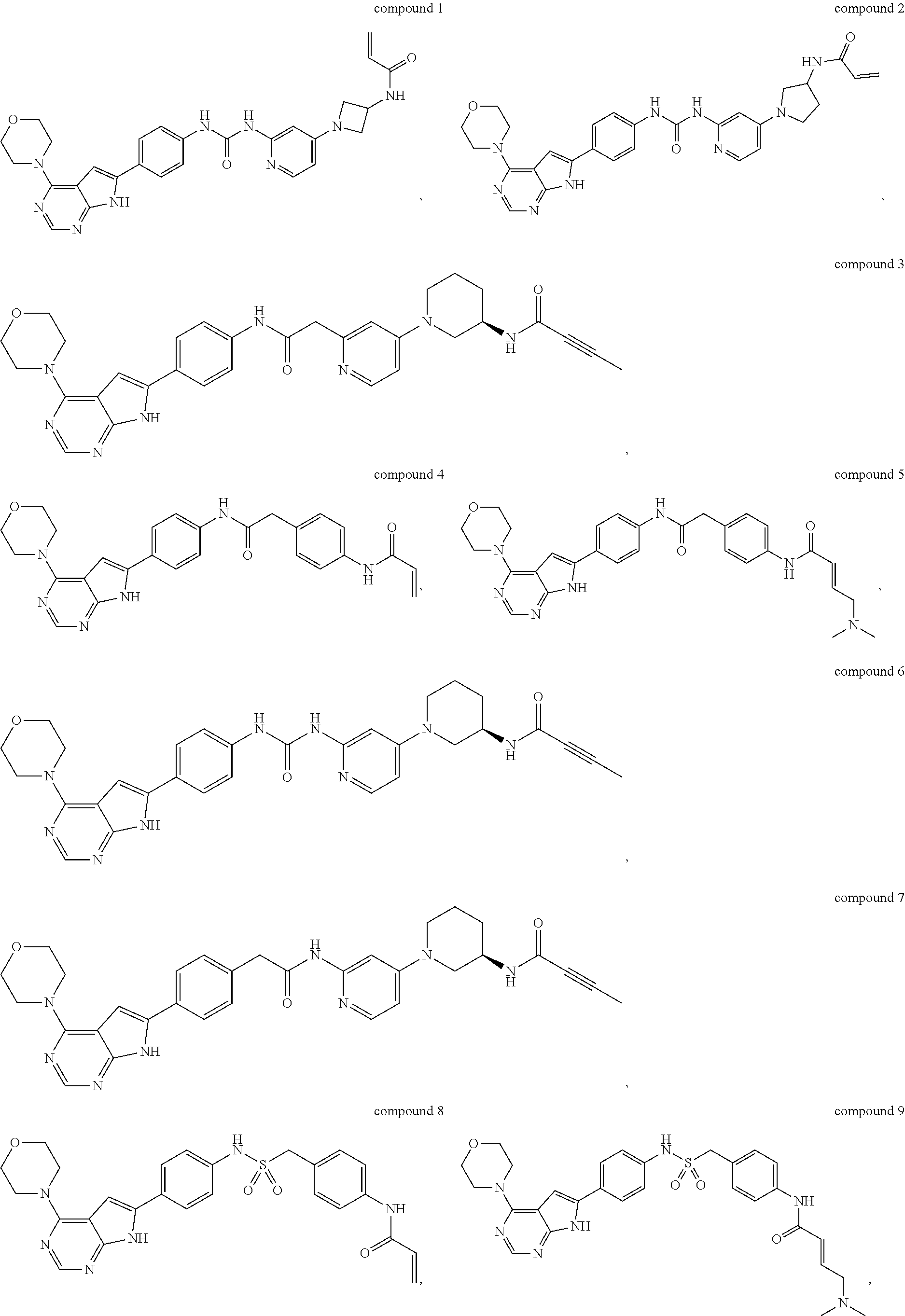

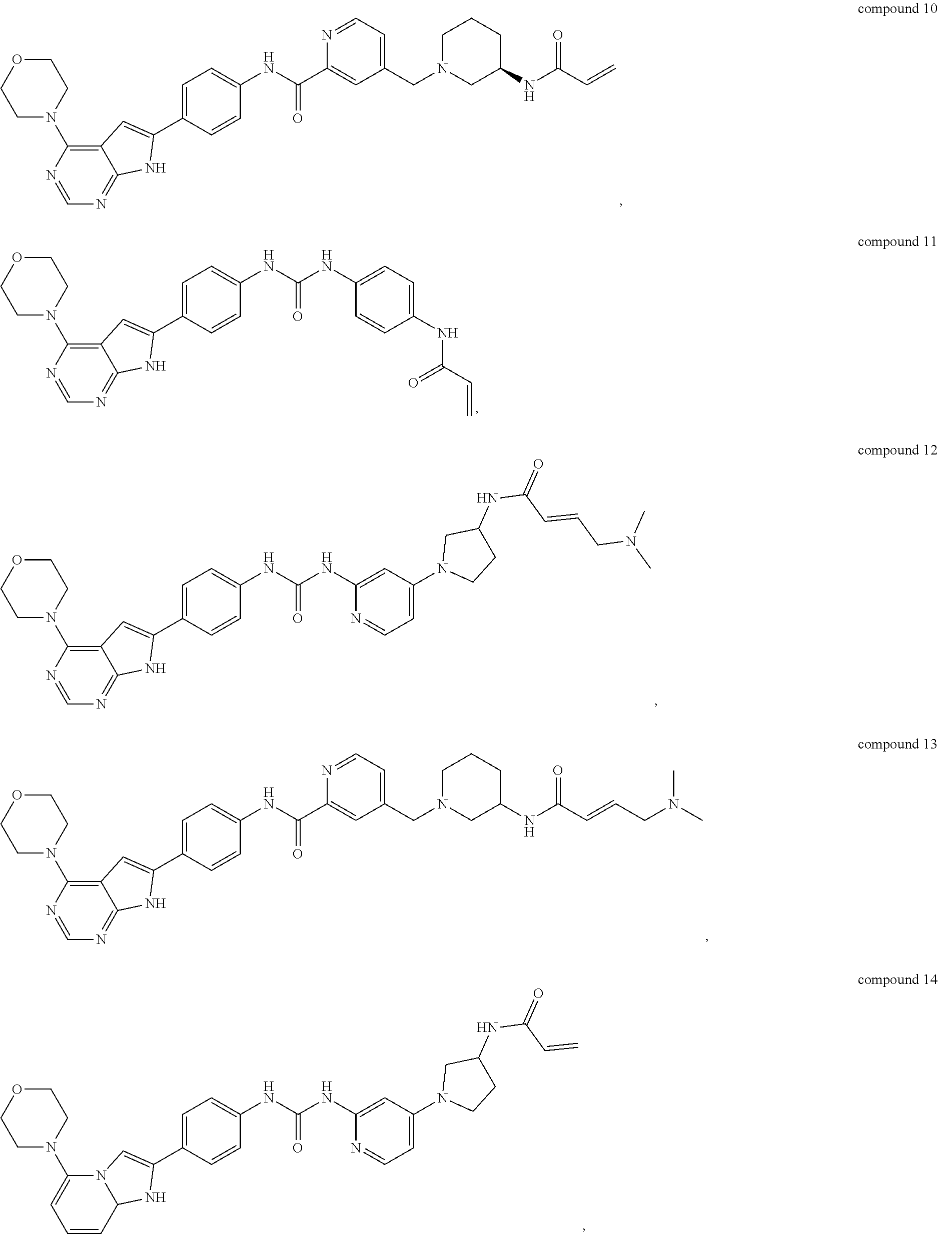 [SMILES: C=CC(=O)N[C@@H]1CCCN(Cc2ccnc(C(=O)Nc3ccc(-c4cc5c(N6CCOCC6)ncnc5[nH]4)cc3)c2)C1 ; C=CC(=O)Nc1ccc(NC(=O)Nc2ccc(-c3cc4c(N5CCOCC5)ncnc4[nH]3)cc2)cc1 ; CN(C)C/C=C/C(=O)NC1CCN(c2ccnc(NC(=O)Nc3ccc(-c4cc5c(N6CCOCC6)ncnc5[nH]4)cc3)c2)C1 ; CN(C)C/C=C/C(=O)NC1CCCN(Cc2ccnc(C(=O)Nc3ccc(-c4cc5c(N6CCOCC6)ncnc5[nH]4)cc3)c2)C1 ; C=CC(=O)NC1CCN(c2ccnc(NC(=O)Nc3ccc(C4=CN5C(N6CCOCC6)=CC=CC5N4)cc3)c2)C1]

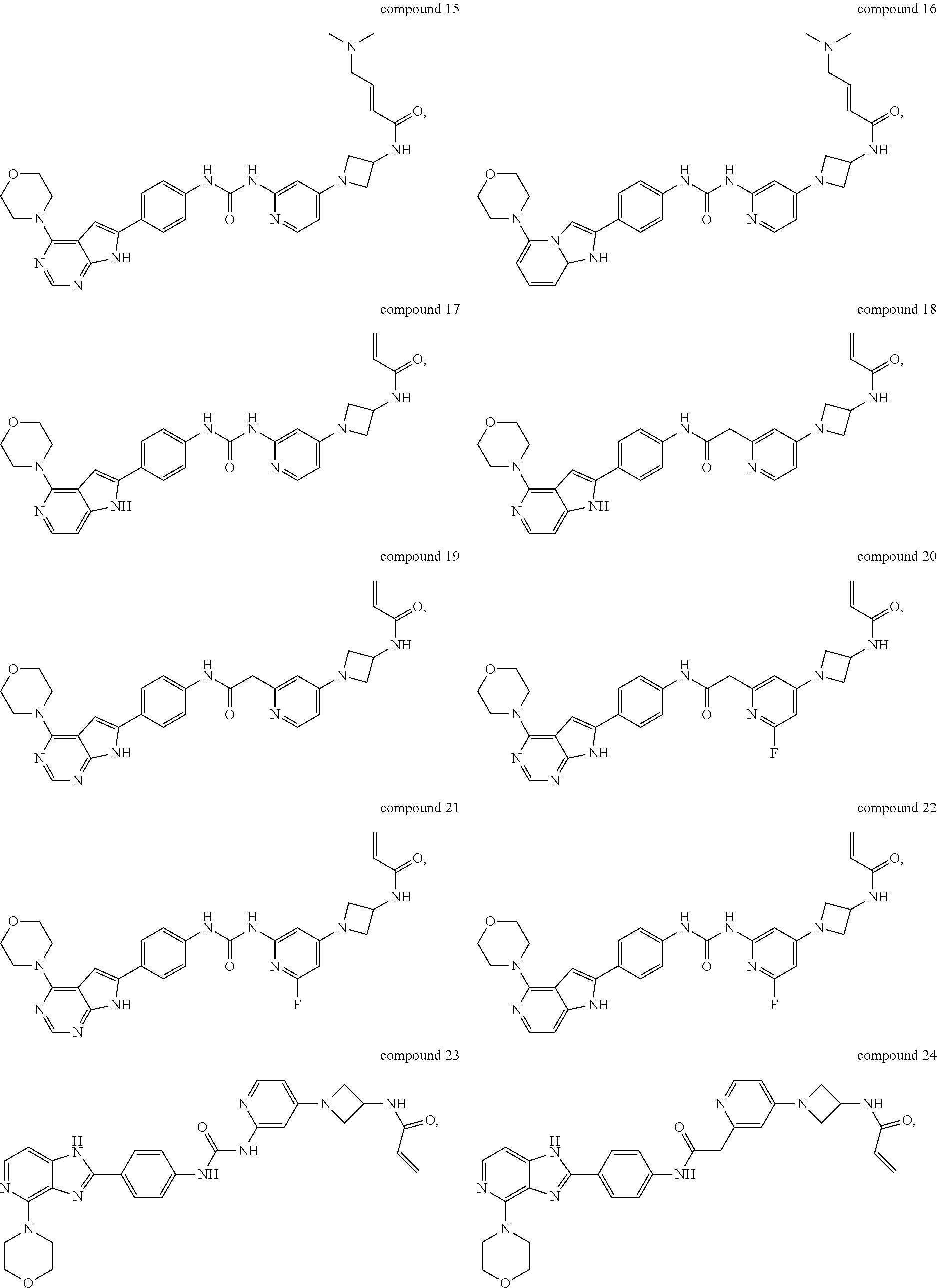

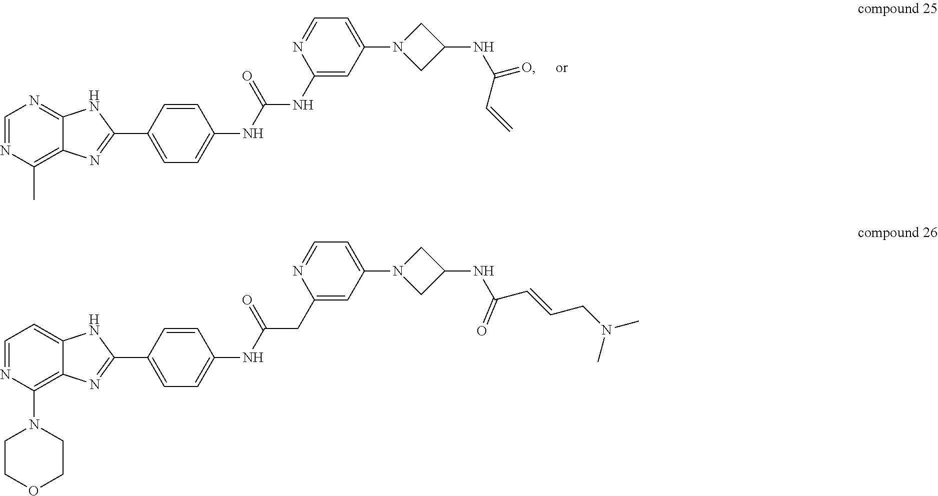 [SMILES: C=CC(=O)NC1CN(c2ccnc(NC(=O)Nc3ccc(-c4nc5c(C)ncnc5[nH]4)cc3)c2)C1 ; CN(C)C/C=C/C(=O)NC1CN(c2ccnc(CC(=O)Nc3ccc(-c4nc5c(N6CCOCC6)nccc5[nH]4)cc3)c2)C1]

20. The method of claim 1, further comprising: inducing improved beta cell function.

21. The method of claim 20, wherein improved beta cell function comprises an increase in beta cell insulin content, glucose-stimulated insulin secretion, or both.

22. A composition comprising expanded pancreatic beta cells produced by the method of claim 1.

23. The composition claim 22, comprising at least 10⁶, 10⁷, 10⁸, 10⁹, 10¹⁰, or 10¹¹ expanded pancreatic beta cells.
